SEQUENCE LISTING (17 sequences)

SEQ ID NO: 1 - protein, 10 aa, Homo sapiens
VVALLGFGYR

SEQ ID NO: 2 - DNA, 30 bp, Artificial Sequence
  Primer
gctagaattcatgatgtctcgtttaaagac

SEQ ID NO: 3 - DNA, 30 bp, Artificial Sequence
  Primer
ggatctcgagatgatgtctcgtttaaagac

SEQ ID NO: 4 - DNA, 26 bp, Artificial Sequence
  Primer
cgtagtcgactaggtccctcggtacc

SEQ ID NO: 5 - DNA, 30 bp, Artificial Sequence
  Primer
aactgccgcggttagatagacggatcattt

SEQ ID NO: 6 - DNA, 46 bp, Artificial Sequence
  Primer
atggactacaaggacgacgatgacaagtctcgtttaaagacggccg

SEQ ID NO: 7 - DNA, 26 bp, Artificial Sequence
  Primer
cgtagtcgactaggtccctcggtacc

SEQ ID NO: 8 - protein, 25 aa, Myxoma poxvirus
KISVYLTAAVVGFVAYGILKWYRGT

SEQ ID NO: 9 - protein, 22 aa, Herpesvirus
KTLLSLALVGACITLGAYLSHK

SEQ ID NO: 10 - protein, 22 aa, Herpesvirus
RWFLTGMTVAGVVLLGSLFSRK

SEQ ID NO: 11 - protein, 23 aa, Herpesvirus
RLLIQAFLSGFFATAIFFIWKRL

SEQ ID NO: 12 - protein, 30 aa, Herpesvirus
RWSMIGAGVTAGAIGIVGVVVCGRMMFSLK

SEQ ID NO: 13 - protein, 29 aa, Herpesvirus
RFSWTLFLAGLTLSLLVICSYLFISRGRH

SEQ ID NO: 14 - protein, 24 aa, Human herpesvirus 8
RMTALLGSIALLATILAAVAMSRR

SEQ ID NO: 15 - protein, 32 aa, Homo sapiens
KVFLPSLLLSHLLAIGLGIYIGRRLTTSTSTF

SEQ ID NO: 16 - protein, 30 aa, Homo sapiens
KVFIPSLFLSHVLALGLGIYIGKRLSTPSA

SEQ ID NO: 17 - protein, 211 aa, Homo sapiens
MASGQGPGPPRQECGEPALPSASEEQVAQDTEEVFRSYVFYRHQQEQEAEGVAAPADPEMVTLPLQPSSTMGQVGRQLAIIGDDINRRYDSEFQTMLQHLQPTAENAYEYFTKIATSLFESGINWGRVVALLGFGYRLALHVYQHGLTGFLGQVTRFVVDFMLHHCIARWIAQRGGWVAALNLGNGPILNVLVVLGVVLLGQFVVRRFFKS

(19) United States
(12) Patent Application Publication
(10) Pub. No.: US 2003/0170722 A1
(43) Pub. Date: Sep. 11, 2003

(54) Methods and reagents for regulating apoptosis

(76) Inventors: Grant McFadden, London (CA); Michael Moran, Toronto (CA)

Correspondence Address: ROPES & GRAY LLP, ONE INTERNATIONAL PLACE, BOSTON, MA, 02110-2624, US

(21) Appl. No.: 10/090,440

(22) Filed: Mar. 4, 2002

Related U.S. Application Data
(60) Provisional application No. 60/273,091, filed on Mar. 2, 2001.

(51) Int. Cl. G01N 33/53; G01N 33/574; A61K 35/84; A61K 35/72; A61K 35/78; A61K 35/70

(52) U.S. Cl. 435/7.1; 435/7.23; 424/195.15; 424/725; 424/195.16

(57) ABSTRACT

The present invention relates to methods and reagents useful for inhibition of apoptosis. Specifically, the invention relates to methods to identify agents that is either pro-apoptotic or anti-apoptotic, especially by affecting the signal transduction pathways of BAK protein and/or M11L protein. The invention also provides business methods, diagnostic kits for identification of pro- or anti-apoptotic agents. Another aspect of the invention provides a method to identify M11L-interacting polypeptides using mass spectrometry.

### REFERENCE TO RELATED APPLICATIONS

This application claims priority to U.S. Provisional application 60/273,091, filed on Mar. 2, 2001, the specification of which is hereby incorporated by reference in its entirety.

### BACKGROUND OF THE INVENTION

The term apoptosis first appeared in the biomedical literature to delineate a structurally distinctive mode of cell death. The cardinal morphological features are cell shrinkage, accompanied by bubbling and blebbing from the surface, and culminating in separation of the cell into a cluster of membrane-bounded bodies. Organellar structure is usually preserved intact, but the nucleus undergoes a characteristic condensation of chromatin, initiated at sublamellar foci and often extending to generate toroidal or cap-like, densely heterochromatic regions. Changes in several cell surface molecules also ensure that, in tissues, apoptotic cells are immediately recognized and phagocytosed by their neighbors. The result is that many cells can be deleted from tissues in a relatively short time with little to show for it in conventional microscopic sections.

This process is responsible for cell death in development, normal tissue turnover, atrophy induced by endocrine and other stimuli, negative selection in the immune system, and a substantial proportion of T-cell killing. It also accounts for many cell deaths following exposure to cytotoxic compounds, hypoxia or viral infection. It is a major factor in the cell kinetics of tumors, both growing and regressing. Many cancer therapeutic agents exert their effects through initiation of apoptosis, and even the process of carcinogenesis itself seems sometimes to depend upon a selective, critical failure of apoptosis that permits the survival of cells after mutagenic DNA damage. Apoptosis is also understood to contribute to many chronic degenerative processes, including Alzheimer's disease, Parkinson's disease and heart failure.

Programmed cell death serves as a major mechanism for the precise regulation of cell numbers and as a defense mechanism to remove unwanted and potentially dangerous cells. Despite the heterogeneity of cell death induction pathways, the execution of the death program is often associated with characteristic morphological and biochemical changes, and this form of programmed cell death has been termed apoptosis.

Considerable progress has been made in identifying the molecules that regulate the apoptotic pathway at each level. Of note, both positive and negative regulators, often encoded within the same family of proteins, characterize the extracellular, cell surface and intracellular steps (Oltvai and Korsmeyer (1994) Cell 79:189-192).

One such family of proteins that constitutes an intracellular checkpoint of apoptosis is the BCL-2 family of proteins. The founding member of this family is the apoptosis-inhibiting protein encoded by the bcl-2 proto-oncogene which was initially isolated from a follicular lymphoma (Bakhshi et al., (1985) Cell 41:889-906; Tsujimoto et al, (1985) Science 229:1390-1393; Cleary and Sklar, (1985) PNAS 82:7439-7443). The BCL-2 protein is a 25 kDa, integral membrane protein of the mitochondria. This factor extends survival in many different cell types by inhibiting apoptosis elicited by a variety of death-inducing stimuli (Korsmeyer (1992) Blood 80:879-886).

The family of BCL-2-related proteins has been defined by sequence homology that is largely based upon conserved motifs termed bcl-homology domains. (Yin et al, (1994) Nature 369:321-323). Bcl-homology domains 1 and 2 (BH1 and BH2) domains have been shown to be important in dimerization and in modulating apoptosis (Yin et al., (1994) Nature 369:321-323). A third homology region, BH3, has also been identified as important to dimerization as well as apoptosis (Boyd et al., Oncogene 11:1921-1928; Chittenden et al., (1995) EMBO J 14:5589-5596) as has been a fourth homology region, BH4, near the amino terminal end of some family members (Farrow and Brown, (1996) Curr Opin Genet Dev 6:45-49).

Members of this family can heterodimerize and, in most cases, homodimerize as well. The ratio of death antagonists (BCL-2, BCL-X_{L}, MCL-1 and A1) to agonists (BAX, BAK, BCL-X_{S} and BAD) determines which homodimers or heterodimers are formed and the balance of these is believed to determine whether a cell will respond to an apoptotic signal (Oltvai and Korsmeyer, (1994) Cell 79:189-192). Thus, dimerization between agonists and antagonists is competitive. For example, the death promoting molecule BAX forms homodimers that favor death whereas BAX will also form heterodimers with BCL-2 or BCL-X_{L} (Oltvai et al., (1993) Cell 74:609-619) and the formation of these heterodimers results in inhibition of cell death. Mutagenesis studies have revealed that intact BH1 and BH2 domains of the antagonists (BCL-2, BCL-X_{L}) are required for them to heterodimerize with BAX and to repress cell death (Yin et al., (1994) Nature 369:321-323). Conversely, deletion analysis has indicated that the BH3 domain of death agonists (BAK, BAX) is required for them to heterodimerize with BCL-X_{L} or BCL-2 and to promote cell death (Chittenden et al., (1995) EMBO J 14:5589-5596). However, other mutations in BCL-X_{L} have been noted to disrupt heterodimerization with BAX, but retain death repressor activity (Cheng et al., (1996) Nature 379: 554-556). This suggests that these molecules might also work independent of one another. Recently, the first X-ray and multidimensional NMR structure of a family member, BCL-X_{L}, was determined (Muchmore et al., (1996) Nature 381:335-341). It was found that alpha helices correspond to the BH1-BH4 domains and that a hydrophobic pocket results from the close spatial proximity of the BH1, BH2 and BH3 domains.

The BH3 domain of BAK, an agonist with BH1, BH2 BH3 and C-terminal membrane localization domains, has been postulated to be of central importance in mediating the cell death promoting effect of this family member. This conclusion was based upon deletion studies which identified the BH3 region as necessary for induction of cell-death and upon the retention of cell killing activity by a 50 amino acid polypeptide fragment including BH3 but excluding BH1 and BH2 which indicated that the BH3 domain is sufficient for eliciting cell death.

Some disease conditions are believed to be related to the development of a defective down-regulation of apoptosis in the affected cells. For example, neoplasias may result, at least in part, from an apoptosis-resistant state in which cell proliferation signals inappropriately exceed cell death signals. Furthermore, some DNA viruses such as Epstein-Barr virus, African swine fever virus and Adenovirus, parasitize the host cellular machinery to drive their own replication and at the same time modulate apoptosis to repress cell death and allow the target cell to reproduce the virus. Moreover, certain disease conditions such as lympho-proliferative conditions, cancer including drug resistant cancer, arthritis, inflammation, autoimmune diseases and the like may result from a down regulation of cell death regulation. In such disease conditions it would be desirable to promote apoptotic mechanisms and one advantageous approach might involve treatment with a cell death agonists having a BH3 domain which has been identified as being an important agonist determinant.

Furthermore, in certain disease conditions it would be desirable to inhibit apoptosis such as in the treatment of immunodeficiency diseases, including AIDS, senescence, neuro-degenerative disease, ischemic and reperfusion cell death, infertility, wound-healing, and the like. In the treatment of such diseases it would be desirable to diminish the cell death agonist activity of endogenous proteins containing BH3 domains. Thus it would be desirable to identify new members of the BCL-2 family which have cell-death agonist properties by virtue of the presence of a BH3 domain and to utilize these as a basis for treatment modalities in advantageously modulating the apoptotic process in disease conditions involving either inappropriate repression or inappropriate enhancement of cell death.

### SUMMARY OF THE INVENTION

The present invention relates to the discovery of the mechanism of action by which viruses, particularly poxviruses, are able to suppress apoptosis in infected cells. M11L, for example, is a protein produced by the Myxoma poxvirus that is required to prevent apoptosis of virus-infected leukocytes and also plays an anti-apoptotic role when expressed autonomously from other viral proteins. As described in greater detail below, M11L is targeted to the mitochondria of the host cell and functions upstream of caspase-3 activation and cytochrome c release but downstream of Bid cleavage. This provides evidence that M11L impacts apoptotic cascades by modulating the mitochondrial control point of cell death. This view is further supported by the finding that M11L is localized to the cytoplasmic aspect of mitochondria by a short 25 a.a. C-terminal targeting motif. A point mutation in this motif redirects the M11L variant protein to other intracellular membranes, notably the endoplasmic reticulum. M11L can protect mitochondria from undergoing loss of inner membrane potential, an event frequently associated with apoptosis and experiments to determine how this is achieved are currently in progress.

The present application also describes the discovery that M11L can be found in complexes including BAK, an apoptotic member of the Bcl-2 family. BAK functions to accelerate apoptosis by binding to and antagonizing Bcl-2. These results are consistent with a role for M11L as an antagonist of BAK activity, e.g., inhibiting the pro-apoptotic activity of BAK.

One aspect of the present invention relates to drug screening assays for identifying agents capable of inhibiting M11L activity, e.g., that promote apoptosis of cells in which M11L is expressed. In other embodiments, the assay can be used to identify agents which mimic the activity of M11L by inhibiting the apoptotic activity of BAK, and which are therefore anti-apoptotic agents. Such agents can be proteins, peptides, nucleic acids, carbohydrates, small organic molecules, and natural product extract libraries, such as isolated from animals, plants, fungus and/or microbes. Therapeutic applications of apoptosis manipulation include the treatment of acute and chronic neuro-degenerative diseases, i.e. stroke, Alzheimer's or Huntington's disease by drugs, and sensitization of cancer cells for drug/radiation-induced apoptosis by modulation of survival signals and viral transfer of apoptosis promoting genes.

Moreover, anti-apoptotic viral genes like M11L provide a novel resource with which to provide in vivo recombinant expression systems that resist immune clearance by CTLs and NK cells, and which can be potentially exploited for both gene therapy and vaccine vector development. Such systems can utilize recombinant forms of M11L, as well as small molecules which mimic or inhibit M11L activity.

Accordingly, one aspect of the present invention provides a drug screening assay for identifying agents which may be potentially apoptotic or anti-apoptotic, comprising:

a. providing a reaction mixture including complexes of BAK and/or M11L proteins;

b. contacting the reaction mixture with one or more test compounds;

c. determining if the test compound binds to the complex or increases or decreases the steady state level of the complex.

In preferred embodiments, the assay is repeated for a variegated library of at least 10 different test compounds, even more preferably at least 100, 1,000 or even 10,000 different test compounds. Exemplary compounds which can be screened for activity in the subject assays include peptides, nucleic acids, carbohydrates, small organic molecules, and natural product extract libraries, such as isolated from animals, plants, fungus and/or microbes In certain preferred embodiments, the reaction mixture is a whole cell. In other embodiments, the reaction mixture is a cell lysate or purified protein composition.

In certain embodiments, a test compound which is identified as able to bind to or alter the kinetics of complex formation is further tested, e.g., in whole cells or in vivo, for the ability to inhibit or potentiate apoptosis.

Thus, one aspect of the invention provides a method for identifying agents which may be potentially pro-apoptotic or anti-apoptotic, comprising: A) providing a reaction mixture including complexes of BAK and/or M11L proteins; B) contacting the reaction mixture with one or more test agents; C) determining if the test agent possesses at least one of the following abilities: 1) binding to the complex; 2) increasing or decreasing the steady state level of the complex; 3) affecting an enzymatic activity of the complex; 4) affecting a subcellular localization of the complex.

In one embodiment, the agent is polypeptides, nucleic acids, carbohydrates, small organic molecules, or natural product extract libraries.

In another embodiment, the agent is from natural product extract libraries isolated form animals, plants, fungus, or microbes.

In another embodiment, the method is repeated for a variegated library of at least 10 different members. In a preferred embodiment, the method is repeated for a variegated library of at least 100 different members, more preferably at least 1,000 different members, and most preferably at least 10,000 different members.

In another embodiment, the method further comprises: D) determining if the test agent, which possesses at least one of the abilities of C), is pro-apoptotic or anti-apoptotic. In a preferred embodiment, step D) is carried out in vivo or in whole cells.

In another embodiment, the reaction mixture is a cell-free system. In a preferred embodiment, the cell-free system comprises reconstituted protein mixture of semi-purified proteins. In another preferred embodiment the cell-free system comprises reconstituted protein mixture of highly-purified proteins substantially lacking impurity. In yet another preferred embodiment, at least one member of said complexes of BAK and/or M11L proteins, or the test agent, is immobilized on a solid support. In a most preferred embodiment, the immobilization is effected by chemical cross-linking, by indirect conjugating via an intermediate molecule, or by direct coating of said solid support. In another most preferred embodiment, the intermediate molecule is an antibody or biotin. In another most preferred embodiment, the solid support is microtiter plates, microarrays, test tubes, microcentrifuge tubes, or solid matrices. In another most preferred embodiment, said at least one member of said complexes of BAK and/or M11L proteins, or said test agent is a fusion protein adapted to bind said solid support. In another embodiment, at least one member of said complexes of BAK and/or M11L proteins, or the test agent, is labeled. In a preferred embodiment, the label is a radioisotope, a fluorescent label/tag, an epitope tag, or an enzyme.

In another embodiment, the cell-free system is generated from lysates, each containing one or more of the relevant polypeptides, which lysates are mixed appropriately or spiked, wherein no single lysate contains all the component necessary for generating said cell-free system. In a preferred embodiment, one or more of said relevant polypeptides is recombinantly generated. In another preferred embodiment, said lysates derive from one or more cell types selected from bacteria cells, yeast cells, worm cells, insect cells, amphibian cells, plant cells, or mammalian cells.

In another embodiment, the reaction mixture is a cell. In a preferred embodiment, the method is carried out using a yeast two-hybrid assay or reverse yeast two-hybrid assay. In a most preferred embodiment, the method employs an Interaction Trap System (ITS) or reverse ITS.

Still another aspect of the present invention provides a method of conducting a drug discovery business comprising: A) providing one or more assay systems for identifying agents by their ability to inhibit or potentiate BAK-dependent and/or M11L-dependent apoptosis; B) conducting therapeutic profiling of agents identified in step A), or further analogs thereof, for efficacy and toxicity in animals; and C) formulating a pharmaceutical preparation including one or more agents identified in step (B) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Yet another aspect of the invention provides a method of conducting a target discovery business comprising: A) providing one or more assay systems for identifying agents by their ability to inhibit or potentiate BAK-dependent and/or M11L-dependent apoptosis; B) (optionally) conducting therapeutic profiling of agents identified in step A) for efficacy and toxicity in animals; and C) licensing, to a third party, the rights for further drug development and/or sales for gents identified in step A), or analogs thereof.

Another aspect of the invention provides a method to identify M11L-interacting polypeptides, comprising: A) contacting M11L with a reaction mixture; B) retrieving M11L, together with any M11L-interacting polypeptides, from the reaction mixture; C) identifying M11L-interacting polypeptides of B) using mass spectrometry.

In one embodiment, M11L is a recombinant protein.

In another embodiment, M11L is provided as a fusion protein.

In another embodiment, the reaction mixture is a cell-free system.

In another embodiment, the reaction mixture is a cell.

In another embodiment, step B) is effected by immunoprecipitation.

In another embodiment, the method further comprises separating M11L from M11L-interacting polypeptides before step C). In a preferred embodiment, the method further comprises digesting separated M11L-interacting polypeptides before step C).

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### BRIEF DESCRIPTION OF DRAWINGS

FIG. 1. M11L is antiapoptotic. (A) Mock or myxoma virus (vMyxlac)infected RL-5 rabbit T lymphocytes were treated with 2 μM staurosporine, and apoptosis was measured using TUNEL analysis at the time intervals indicated. Levels of TUNEL-positive (apoptotic) cells were elevated in the mock-infected cell population compared with myxoma virus-infected cells, indicating that virus infection protects RL-5 cells from apoptosis after staurosporine treatment. (B) Rat2 fibroblasts ectopically expressing M11L (Rat2M11L) or containing the vector alone (Rat2puro) were treated with 2 μM staurosporine, and apoptosis was monitored at the times indicated using TUNEL analysis. Apoptosis levels were elevated in Rat2puro cells compared with Rat2M11L cells, indicating that M11L expression alone protects Rat2 cells from the proapoptotic effects of staurosporine. (C) Rat2puro and Rat2M11L cells were treated with 5 μM staurosporine for the times indicated, and Caspase-3 was detected in whole cell lysates by SDS-PAGE and immunoblot analysis using an antibody directed against the large subunit of the active caspase. Cleavage of the 32 kD proCaspase-3 to produce the detectable 19 kD component of the active caspase was observed in Rat2puro cells (top) but was considerably reduced in Rat2M11L cells (bottom). Hence, M11L expression impedes Caspase-3 activation after treatment of Rat2 cells with staurosporine.

FIG. 2. M11L localizes to mitochondria in infected cells. (A) BGMK cells infected for 20 h with M11L-expressing myxoma virus (vMyxlac) or the M11L knockout virus (vMyxM11L⁻) were treated with Mitotracker Red to identify mitochondria, and M11L was detected by indirect immunofluorescence. Cells were visualized by confocal microscopy. As expected, M11L was detected in cells infected with vMyxlac (a) but not in cells infected with the M11L knockout virus (d), and Mitotracker Red produced punctate mitochondrial staining (b and e). Superimposed Mitotracker Red and M11L signals (c) yielded a yellow image, indicating that M11L localizes to mitochondria. This was not observed in cells infected with the knockout virus (f). Bar, 10 nm. (B) The proteinase K (PK) sensitivity of the 18 kD M11L (top) or 17 kD COX IV (bottom) proteins was assessed. Digitonin lysates of HepG2 cells infected with M11L-expressing VVM11L or the control virus VV601 (CNTL) were prepared 12 h after infection. Pellet (lanes 1, 2, 5, and 6) and supernatant (sup; lanes 3, 4, 7, and 8) fractions were isolated. Samples were subjected to proteinase K treatment for 20 min (PK 20 min; lanes 1-4) or left untreated (PK 0 min; lanes 5-8), and M11L or COX IV were detected by SDS-PAGE and immunoblotting. M11L (top) but not COX IV (bottom) in the pellet fraction (lane 1) was sensitive to proteinase K treatment, indicating that although M11L is membrane associated, it is orientated towards the cytosol.

FIG. 3. GFP-tagged M11L localizes to mitochondria. COS-7 cells expressing GFP alone (a), M11L bearing an NH2 terminal GFP tag (d), or a GFP-tagged, truncated form of M11L (GFP-M11Lstop) lacking the last 24 amino acids including the hydrophobic region (g) were visualized by confocal microscopy. Mitochondria were identified by Mitotracker Red staining (b, e, and h). When the Mitotracker Red fluorescence signal was merged with that of GFP-M11L, a yellow image was produced (f), indicating that GFP-M11L localizes to mitochondria in live, transfected cells. In contrast, no colocalization was observed in the case of GFP alone (c) or truncated M11L (i). Failure of truncated M11L to localize to mitochondria indicates that the last 24 amino acids are necessary for targeting. Bar, 10 nm.

FIG. 4. M11L contains a COOH terminal mitochondrial targeting signal. COS-7 or HeLa cells expressing GFP-mt, a construct consisting of GFP tagged with the COOH terminal 25 amino acids of M11L (mt) containing the putative transmembrane domain (underlined), were visualized by confocal microscopy. The distribution of the GFP-mt (a and d) and Mitotracker red (b and e) was found to be coincident (c and f). Hence, the COOH terminal 25 amino acids of M11L are sufficient for mitochondrial targeting. Bars, 10 nm.

FIG. 5. The M11L mitochondrial targeting signal belongs to a consensus found in other proteins. Proposed COOH terminal consensus for targeting Bcl-2 family members to the mitochondrial outer membrane. The COOH terminal sequences shown include those of the antiapoptotic Bcl-2 family members Bcl-2, BClX_{L}, Boo/Diva, and CED-9, the viral antiapoptotic proteins M11L, BHRF1, and KSbcl-2, as well as the proapoptotic proteins Nip3 and Nix.

FIG. 6. M11L prevents the mitochondrial permeability transition. (A) Rat2puro (top) or Rat2M11L (bottom) cells were maintained as controls or treated with staurosporine for 4 h and stained with the fluorescent dye DiOC6 to obtain a measure of the mitochondrial membrane potential. Representative results of one of three separate experiments are shown. Control cells (a and c) displayed intense staining with the dye, indicating normal mitochondrial function. The protonophore CCCP markedly attenuated the fluorescent signal, as expected (a, insert). A similar reduction in the fluorescent signal was seen in Rat2puro cells after apoptosis induction by staurosporine (b). In contrast, signal intensity and, therefore, mitochondrial function was retained in Rat2M11L cells after the same treatment (d). This shows that M11L plays a role in preserving mitochondrial function after apoptosis induction. (B) HeLa cells were transiently transfected to allow expression of GFP, mitochondria-targeted GFP (GFP-mt), or the fusion proteins GFP-M11L, GFP-M11L stop, or GFP-Bcl-2. The percentage of GFP-expressing cells that also displayed TMRE fluorescence was determined with or without staurosporine treatment. The reduction in the percentage of TMRE-positive cells after staurosporine treatment is represented here graphically and is the average of two separate experiments. The results show that a large percentage of cells expressing GFP, GFP-mt, and the GFP-M11L stop chimera (which is not localized to mitochondria) failed to retain TMRE fluorescence after staurosporine treatment. CCCP also produced a loss of TMRE fluorescence in GFP-expressing cells, as expected. In contrast, GFP-M11L and GFP-Bcl-2 expressing cells maintained TMRE fluorescence after staurosporine treatment. This indicates that M11L, like Bcl-2, can protect the mitochondria of HeLa cells from undergoing loss of membrane potential after apoptosis induction.

FIG. 7. M11L is required to prevent apoptosis during myxoma virus infection of primary rabbit monocytes. Primary rabbit monocytes were infected with the M11L knockout virus (vMyxM11L³¹ ), a revertant of this knockout virus (vMyxM11L^{R}), control (vMyxlac), or three other myxoma virus constructs with targeted gene disruptions (vMyxT2 2, vMyxT4 2, and vMyxSerp 2). Apoptosis was measured by TUNEL analysis (horizontal axis), and the CD11b positive cells of monocyte origin were identified by indirect immunofluorescence (vertical axis). Apoptotic monocytes are represented in the second quadrant (percentage of total cells shown). Apoptosis levels were elevated in cells infected with the M11L knockout virus (vMyxM11L⁻) but not in cells infected with the other virus variants, all of which express M11L. The data shown are representative of four separate experiments and demonstrate a distinct role for M11L in preventing apoptosis of infected monocytes.

FIG. 8. Flow diagram illustrating a CaCl₂/BES transfection method. In the illustrated embodiment, the Gel is a 4-15% Tris-HCl gradient gel from BioRad, and the stain is Pierce Gelcode Blue (colloidal coomassie).

FIG. 9. Gel of immunoprecipitated products. The first lane is molecular weight markers. The second lane is an IP from untransfected HEK293T cells. The third lane is an IP from cells transfected with Flag-M11L. Each of the bands in the second and third lane, were excised, digested with trypsin and analyzed by mass spec.

FIG. 10. MS spectra for a peptide analyzed by nanospray mass spectrometer in MS/MS mode. The amino acids are read from right to left and are determined from the mass difference between the ions shown.

FIG. 11. Amino acid sequence of BAK. The peptide shown on in FIG. 10 was VVALLGFGYR (SEQ ID No. 1) and it was one of three peptides (shown here in bold) identified by nanospray mass spectrometry from the protein BAK. These three peptides are sufficient to confirm that BAK was the protein isolated from one of the bands analyzed. Other bands excised from the gel were analyzed by mass spectrometry in a similar fashion.

### DETAILED DESCRIPTION OF THE INVENTION

I. Definitions

For convenience, certain terms employed in the specification, examples, and appended claims are collected here.

“Apoptosis” (or “normal” or “programmed” cell death) is the physiological process by which unwanted or useless cells are eliminated during development and other normal biological processes.

Apoptosis, is a mode of cell death that occurs under normal physiological conditions and the cell is an active participant in its own demise (“cellular suicide”). It is most often found during normal cell turnover and tissue homeostasis, embryogenesis, induction and maintenance of immune tolerance, development of the nervous system and endocrine-dependent tissue atrophy.

Cells undergoing apoptosis show characteristic morphological and biochemical features. These features include chromatin aggregation, nuclear and cytoplasmic condensation, partition of cytoplasm and nucleus into membrane bound vesicles (apoptotic bodies) which contain ribosomes, morphologically intact mitochondria and nuclear material. In vivo, these apoptotic bodies are rapidly recognized and phagocytized by either macrophages or adjacent epithelial cells. Due to this efficient mechanism for the removal of apoptotic cells in vivo no inflammatory response is elicited. In vitro, the apoptotic bodies as well as the remaining cell fragments ultimately swell and finally lyse. This terminal phase of in vitro cell death has been termed “secondary necrosis.”

“Anti-apoptotic” or “pro-apoptotic” refers to the ability of an agent to suppress or promote apoptosis, particularly when comparing the same system (in vitro or in vivo assay conditions) with or without a test agent. Thus, a control assay at the absence of the test agent is usually performed to provide a baseline for comparison.

The term “agent” refers broadly to molecules such as proteins, peptides, nucleic acids, carbohydrates, small organic molecules, or natural product extract libraries, such as isolated from animals, plants, fungus and/or microbes. When referring to a nucleic acid, it means a nucleic acid which, itself, its transcriptional product and/or translation product thereof have some biological activity in a cell. Thus, in that case, the term includes coding sequences for polypeptides, antisense constructs, decoy constructs, etc.

As used herein the term “animal” refers to mammals, preferably mammals such as humans. Likewise, a “patient” or “subject” to be treated by the method of the invention can mean either a human or non-human animal.

As used herein, “Baks” or “Bak” refers to the nucleic acid molecules described herein (Bak and Bak-2 derivatives thereof), “the BAKs” or “BAK” refers to the proteins encoded thereby (BAK, BAK-2 and derivatives thereof). The Bak nucleotides include, but are not limited to, the cDNA, genome-derived DNA and synthetic or semi-synthetic DNA or RNA. The nucleotide sequence of a human Bak cDNA is provided by GenBank No. NM001188.

As used herein, the term “cellular composition” refers to a preparation of cells, which preparation may include, in addition to the cells, non-cellular components such as cell culture media, e.g. proteins, amino acids, nucleic acids, nucleotides, co-enzyme, anti-oxidants, metals and the like. Furthermore, the cellular composition can have components which do not affect the growth or viability of the cellular component, but which are used to provide the cells in a particular format.

“Cytotoxicity” is the cell-killing property of a chemical compound (such as a food, cosmetic, or pharmaceutical) or a mediator cell (cytotoxic T cell). In contrast to necrosis and apoptosis, the term cytotoxicity does not indicate a specific cellular death mechanism. For example, cell-mediated cytotoxicity (that is, cell death mediated by either cytotoxic T lymphocytes [CTL] or natural killer [NK] cells) combines some aspects of both necrosis and apoptosis.

“Expression vector” refers to a replicable DNA construct used to express DNA which encodes the desired protein and which includes a transcriptional unit comprising an assembly of (1) agent(s) having a regulatory role in gene expression, for example, promoters, operators, or enhancers, operatively linked to (2) a DNA sequence encoding a desired protein (in this case, a MIF protein of the present invention) which is transcribed into mRNA and translated into protein, and (3) appropriate transcription and translation initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

In the expression vectors, regulatory elements controlling transcription or translation can be generally derived from mammalian, microbial, viral or insect genes. The ability to replicate in a host, usually conferred by an origin of replication, and a selection gene to facilitate recognition of transformants may additionally be incorporated. Vectors derived from viruses, such as retroviruses, adenoviruses, and the like, may be employed.

As used herein, the term “fusion protein” is art recognized and refer to a chimeric protein which is at least initially expressed as single chain protein comprised of amino acid sequences derived from two or more different proteins, e.g., the fusion protein is a gene product of a fusion gene.

As used herein, the term “gene” or “recombinant gene” refers to a nucleic acid comprising an open reading frame encoding a polypeptide, including both exonic and (optionally) intronic sequences.

The “growth state” of a cell refers to the rate of proliferation of the cell and the state of differentiation of the cell.

“Homology” and “identity” each refer to sequence similarity between two polypeptide sequences, with identity being a more strict comparison. Homology and identity can each be determined by comparing a position in each sequence which may be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. An “unrelated” or “non-homologous” sequence shares less than 40 percent identity, though preferably less than 25 percent identity.

As used herein, an “indicator gene construct” is a nucleic acid that includes a “indicator gene” operatively linked to at least one transcriptional regulatory sequence. Transcription of the indicator gene is controlled by these sequences to which they are linked. The activity of at least one or more of these control sequences can be directly or indirectly regulated by the reporter protein upon release from the mitochondria. Exemplary transcriptional control sequences are promoter sequences. An indicator gene is meant to include a promoter-indicator gene construct which is heterologously expressed in a cell.

As used herein, “immortalized cells” refers to cells which have been altered via chemical and/or recombinant means such that the cells have the ability to grow through an indefinite number of divisions in culture.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

As used herein, “proliferating” and “proliferation” refer to cells undergoing mitosis.

A “protein coding sequence” or a sequence which “encodes” a particular polypeptide or peptide, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

However, as described below, the generic term “coding sequence” may refer to, as the context permits, sequences which are transcribed to produce RNA which is itself directly active (as a potential agent), as opposed to a polypeptide translated therefrom.

Likewise, “encodes,” unless evident from its context, will be meant to include DNA sequences which encode a polypeptide, as the term is typically used, as well as DNA sequences which are transcribed into inhibitory antisense molecules.

“Substantially lacking impurity” as used herein means the percentage (by weight or volumn) of impurity is at least less than 25%, more preferably less than 20%, 15%, 10%, 5%, 3%, 1%, most preferably less than 0.1% or 0.01%. “Impurity” means any component or composition that is not intended to be present, or not known to be present, in certain mixtures.

As used herein, the term “transfection” means the introduction of a heterologous nucleic acid, e.g., an expression vector, into a recipient cell by nucleic acid-mediated gene transfer. “Transformation”, as used herein with respect to transfected nucleic acid, refers to a process in which a cell's genotype is changed as a result of the cellular uptake of exogenous DNA or RNA, and, for example, the transformed cell expresses a recombinant form of a polypeptide, e.g., a reporter protein of the present invention.

“Transient transfection” refers to cases where exogenous DNA does not integrate into the genome of a transfected cell, e.g., where episomal DNA is transcribed into mRNA and translated into protein.

A cell has been “stably transfected” with a nucleic acid construct when the nucleic acid construct is capable of being inherited by daughter cells.

“Transcriptional regulatory sequence” is a generic term used throughout the specification to refer to DNA sequences, such as initiation signals, enhancers, and promoters and the like which induce or control transcription of protein coding sequences with which they are operably linked. It will be understood that a recombinant gene can be under the control of transcriptional regulatory sequences which are the same or which are different from those sequences which control transcription of the naturally-occurring form of the gene, if any.

As used herein, the term “tissue-specific promoter” means a DNA sequence that serves as a promoter, i.e., regulates expression of a selected DNA sequence operably linked to the promoter, and which effects expression of the selected DNA sequence in specific cells of a tissue, such as cells of a urogenital origin, e.g. renal cells, or cells of a neural origin, e.g. neuronal cells. The term also covers so-called “leaky” promoters, which regulate expression of a selected DNA primarily in one tissue, but cause expression in other tissues as well.

“Operably linked” when describing the relationship between two DNA regions simply means that they are functionally related to each other. For example, a promoter or other transcriptional regulatory sequence is operably linked to a coding sequence if it controls the transcription of the coding sequence.

As used herein, “transformed cells” refers to cells which have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control.

II. Illustrative Embodiments

A. Drug Screening Assays

The present invention provides a systematic and practical approach for the identification of candidate agents able to inhibit M11L activity, e.g., that promote apoptosis of cells in which M11L is expressed, or to mimic the activity of M11L by inhibiting the apoptotic activity of BAK. Such pro-apoptotic and anti-apoptotic agents can be proteins, peptides, nucleic acids, carbohydrates, small organic molecules, and natural product extract libraries, such as isolated from animals, plants, fungus and/or microbes.

In a general sense, certain embodiments of the assays of the present invention evaluate the ability of a compound to modulate (i) protein complexes which include a BAK protein, such as BAK-M11L complexes; (ii) the enzymatic activity, if any, of such multiprotein complexes; or (iii) the cellular localization of such complexes. Merely to illustrate, the assays may be formatted to evaluate the ability of a compound to modulate binding between a BAK protein and another protein (a “target protein”), whether the BAK protein is acting as a subunit of a multiprotein complex or as a substrate for modification.

In other embodiments of the assays, it is the ability of a compound to mimic M11L inhibition of BAK-mediated apoptosis which is tested for. Merely to illustrate, the assays may be formatted to evaluate the ability of a compound to bind to BAK and competitively inhibit binding by M11L. A compound having that primary activity can then be tested to see if it can mimic the anti-apoptotic activity of M11L.

Exemplary compounds which can be screened for activity in the present assays include peptides, nucleic acids, carbohydrates, small organic molecules, and natural product extract libraries, such as isolated from animals, plants, fungus and/or microbes.

There are a variety of assay formats for testing compounds for appropriate apoptotic or anti-apoptotic activity, whether they be peptide or non-peptide. Merely for ease of reading, the following examples are described with respect to the role of M11L in BAK complexes. It will be understood that other protein complexes including BAK and or M11L can be targeted for drug screening assays using similar assays.

The particular assay format selected will reflect the desire to identify compounds which disrupt protein-protein interactions and thereby alter the BAK/M11L complex, or which disrupt the interaction of the complex with, and chemical alteration of a given substrate of the acetylation or other enzymatic activity of the complex, or which disrupt the interaction of the complex with other bcl-2 proteins.

As described herein, inhibitors or mimetics of a BAK bioactivity refer generally to those agents which may act anywhere along the BAK-dependent apoptotic pathway, e.g., from disrupting the interaction of BAK-containing complexes to inhibitors of post-translational modification of BAK, to inhibitors of BAK-dependent membrane permeability.

In general, candidate inhibitors or mimetics of BAK will be screened for activity in appropriate human assays. Compounds that display desired characteristics in a given assay may serve as lead compounds for the discovery of more potent inhibitors. Compounds selected based on their activity in vitro will be screened subsequently in vivo.

(i) Cell-Free Assay Formats

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins or cell-lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with upstream or downstream elements. Accordingly, in an exemplary screening assay of the present invention, a reaction mixture is generated to include a BAK polypeptide, compound(s) of interest, and one or more “target polypeptides”, e.g., proteins, which interacts with the BAK polypeptide. Exemplary target polypeptides include M11L and Bcl-2. Detection and quantification of the formation of complexes including the BAK protein provides a means for determining a compound's efficacy at inhibiting (or potentiating) the bioactivity of BAK. The efficacy of the compound can be assessed by generating dose response curves from data obtained using various concentrations of the test compound. Moreover, a control assay can also be performed to provide a baseline for comparison.

In one embodiment, the subject drug screening assay comprises a reconstituted protein mixture of at least semi-purified proteins. By semi-purified, it is meant that the proteins utilized in the reconstituted mixture have been previously separated from other cellular proteins. For instance, in contrast to cell lysates, the proteins involved in the BAK complex, together with the BAK protein, are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins which might interfere with or otherwise alter the ability to measure the level of BAK complexes.

Complex formation between the BAK polypeptide and a “target polypeptide” (e.g., a protein or protein complex which binds to the BAK polypeptide) may be detected by a variety of techniques. Modulation of the formation of complexes can be quantitated using, for example, detectably labeled proteins such as radiolabelled (e.g. ³²P, ³⁵S, ¹⁴C or ³H), fluorescently labeled (e.g. FITC), or enzymatically labeled BAK polypeptides, by immunoassay, by chromatographic detection, or by detecting the intrinsic activity of either the BAK or target polypeptide. The use of enzymatically labeled proteins will, of course, generally be used only when enzymatically inactive portions of those proteins are used.

Typically, it will be desirable to immobilize either the BAK or the target polypeptide to facilitate separation of complexes from uncomplexed forms of one or both of the proteins, as well as to accommodate automation of the assay. Binding of a BAK polypeptide to the target polypeptide, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/BAK (GST/BAK) fusion proteins can be adsorbed onto glutathione sepharose beads (Sigma Chemical, St. Louis, Mo.) or glutathione derivatized microtitre plates, which are then combined with a preparation of a target polypeptide, e.g. a labeled target polypeptide, along with the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and labeled target polypeptide retained on the matrix determined directly, or in the supernatant after the complexes are subsequently dissociated. Alternatively, the complexes can be dissociated from the matrix, separated by SDS-PAGE, and the level of target polypeptide found in the bead fraction quantitated from the gel using standard electrophoretic techniques.

Other techniques for immobilizing proteins on matrices are also available for use in the subject assay. For instance, either the BAK or target polypeptide can be immobilized utilizing conjugation of biotin and streptavidin. For instance, biotinylated BAK molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with BAK, but which do not interfere with the interaction between the BAK and target polypeptide, can be derivatized to the wells of the plate, and BAK trapped in the wells by antibody conjugation. As above, preparations of a target polypeptide and a test compound are incubated in the BAK-presenting wells of the plate, and the amount of complex trapped in the well can be quantitated. Other exemplary methods for detecting such complexes, in addition to those described above, include detection of a radiolabel or fluorescent label; immunodetection of complexes using antibodies reactive with the target polypeptide, or which are reactive with BAK protein and compete with the target polypeptide; as well as enzyme-linked assays which rely on detecting an enzymatic activity associated with the target polypeptide, e.g., either intrinsic or extrinsic activity.

In the instance of the latter, the enzyme can be chemically conjugated or provided as a fusion protein with the target polypeptide. To illustrate, the target polypeptide can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the target polypeptide and glutathione-S-transferase can be provided, and complex formation quantitated by detecting the GST activity using 1-chloro-2,4-dinitrobenzene (Habig et al (1974) J Biol Chem 249:7130). Alternatively, using such substrates as described above, an intrinsic activity of the target polypeptide can be used to facilitate detection.

For processes which rely on immunodetection for quantitating one of the proteins trapped in the complex, antibodies against the target protein or BAK protein, can be used. Alternatively, the protein to be detected in the complex can be “epitope tagged” in the form of a fusion protein which includes a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein a system (Pharmacia, NJ).

In other embodiments, the cell-free mixtures can be generated using lysates, e.g., derived from cells expressing one or more of the relevant proteins, and mixed appropriately (or spiked) where no single lysate contains all the components necessary for generating the reaction system. In preferred embodiments, one or more of the components, especially the substrate target, are recombinantly produced in a cell used to generate a lysate, or added by spiking a lysate mixture with a purified or semi-purified preparation of the substrate.

The lysates can be derived from any number of cell types, ranging from bacterial cells to yeast cells to cells from metazoan organisms including insects and mammalian cells. To illustrate, a cell-free test system can be reconstituted by mixing cell lysates derived from insect cells expressing BAK and the target protein which have been cloned into baculoviral expression vectors. The cells can be lysed, and if the BAK and target protein are produced by different sets of cells, cell lysates can be accordingly mixed to produce BAK complexes. The level of protein-protein interaction, or if applicable, the enzymatic activity of the complex, can be assessed. As appropriate, the transfected cells can be cells which lack an endogenous BAK protein, or the target protein, can be chosen to be particularly sensitive to avoiding endogenous activity of the cells which may confound the results.

(ii) Cell-Based Assay Formats

In yet further embodiments, the drug screening assay is derived to include a whole cell expressing a BAK protein, along with one or more of target proteins.

To illustrate, the BAK and target proteins can be used to generate an interaction trap assay (see, U.S. Pat. Nos. 5,283,173, 5,580,736, 5,610,015, and 5,695,941; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the proteins to one and other. For instance, an interaction trap assay, especially a reverse-two hybrid system as described by U.S. Pat. Nos. 5,955,280, and 5,965,368, can be derived to identify compounds which inhibit the interaction of BAK and M11L. Such compounds can be used to treat pox-infected patients.

In particular, the method makes use of chimeric genes which express hybrid proteins. To illustrate, a first hybrid gene comprises the coding sequence for a DNA-binding domain of a transcriptional activator fused in frame to the coding sequence for a “bait” protein, e.g., a BAK protein. The second hybrid protein encodes a transcriptional activation domain fused in frame to a gene encoding a “fish” protein which interacts with the BAK protein, e.g. the target protein sequence. If the bait and fish proteins are able to interact, e.g., form a complex, they bring into close proximity the two domains of the transcriptional activator. This proximity is sufficient to cause transcription of a reporter gene which is operably linked to a transcriptional regulatory site responsive to the transcriptional activator, and expression of the reporter gene can be detected and used to score for the interaction of the bait and fish proteins.

In accordance with the present invention, the method includes providing a host cell, preferably a yeast cell, e.g., Kluyverei lactis, Schizosaccharomyces pombe, Ustilaqo maydis, Saccharomyces cerevisiae, Neurospora crassa, Aspergillus niger, Aspergillus nidulans, Pichia pastoris, Candida tropicalis, and Hansenula polymorpha, though most preferably S cerevisiae or S. pombe. The host cell contains a reporter gene having a binding site for the DNA-binding domain of a transcriptional activator, such that the reporter gene expresses a detectable gene product when the gene is transcriptionally activated. Such activation occurs when the activation domain of the transcriptional activator is brought into sufficient proximity to the DNA-binding domain of a transcriptional activator bound to the regulatory element of the reporter gene. The first chimeric gene may be present in a chromosome of the host cell, or as part of an expression vector.

A first chimeric gene is provided which is capable of being expressed in the host cell. The gene encodes a chimeric protein which comprises (i) a DNA-binding domain that recognizes the responsive element on the reporter gene in the host cell, and (ii) bait protein, such as BAK.

A second chimeric gene is provided which is capable of being expressed in the host cell. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid. The second chimeric gene includes a DNA sequence that encodes a second hybrid protein comprising a transcriptional activation domain fused to a fish protein, or a fragment thereof, which is to be tested for interaction with the bait protein. The fish protein can be M11L, Bcl-2, etc., or appropriate fragments thereof.

Preferably, the DNA-binding domain of the first hybrid protein and the transcriptional activation domain of the second hybrid protein are derived from transcriptional activators having separable DNA-binding and transcriptional activation domains. For instance, these separate DNA-binding binding and transcriptional activation domains are known to be found in the yeast GAL4 protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, λcI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction (see, for example, the Brent et al. PCT publication WO94/10300).

In preferred embodiments, any enzymatic activity associated with the bait or fish proteins is inactivated, e.g., dominant negative mutants of GCN5 and the like can be used. Where the interacting proteins are of the enzyme-substrate relationship, mutation of one or more catalytic residues of the enzyme can provide a mutant protein which retains the ability to bind the substrate but not catalytically convert it to product.

Continuing with the illustrated example, the BAK-mediated interaction, if any, between the first second fusion proteins in the host cell, therefore, causes the activation domain to activate transcription of the reporter gene. The method is carried out by introducing the first chimeric gene and the second chimeric gene into the host cell, and subjecting that cell to conditions under which the first hybrid protein and the second hybrid protein are expressed in sufficient quantity for the reporter gene to be activated. The formation of a complex results in a detectable signal produced by the expression of the reporter gene. Accordingly, the formation of a complex in the presence of a test compound to the level of complex in the absence of the test compound can be evaluated by detecting the level of expression of the reporter gene in each case.

In an illustrative embodiment, Saccharomyces cerevisiae YPB2 cells are transformed simultaneously with a plasmid encoding a GAL4db-BAK fusion and with a plasmid encoding the GAL4ad domain fused to an M11L coding sequence. Moreover, the strain is transformed such that the GAL4-responsive promoter drives expression of a phenotypic marker. For example, the ability to grow in the absence of histidine can depend on the expression of the LacZ gene. When the LacZ gene is placed under the control of a GAL4-responsive promoter, the yeast cell will turn blue in the presence of β-gal if a functional GAL4 activator has been reconstituted through the interaction of BAK and M11L. Thus, a convenient readout method is provided. Other reporter constructs will be apparent, and include, for example, reporter genes which produce such detectable signals as selected from the group consisting of an enzymatic signal, a fluorescent signal, a phosphorescent signal and drug resistance.

A similar method modifies the interaction trap system by providing a “relay gene” which is regulated by the transcriptional complex formed by the interacting bait and fish proteins. The gene product of the relay gene, in turn, regulates expression of a reporter gene, the expression of the latter being what is scored in the modified ITS assay. Fundamentally, the relay gene can be seen as a signal inverter. As set out above, in the standard ITS, interaction of the fish and bait fusion proteins results in expression of a reporter gene. However, where inhibitors of the interaction are sought, a positive readout from the reporter gene nevertheless requires detecting inhibition (or lack of expression) of the reporter gene.

In the inverted ITS system, the fish and bait proteins positively regulate expression of the relay gene. The relay gene product is in turn a repressor of expression of the reporter gene. Inhibition of expression of the relay gene product by inhibiting the interaction of the fish and bait proteins results in concomitant relief of the inhibition of the reporter gene, e.g., the reporter gene is expressed. For example, the relay gene can be the repressor gene under control of a promoter sensitive to the BAK/M11L complex described above. The reporter gene can accordingly be a positive signal, such as providing for growth (e.g., drug selection or auxotrophic relief), and is under the control of a promoter which is constitutively active, but can be suppressed by the repressor protein. In the absence of an agent which inhibits the interaction of the fish and bait protein, the repressor protein is expressed. In turn, that protein represses expression of the reporter gene. However, an agent which disrupts binding of the BAK and the target protein results in a decrease in repressor expression, and consequently an increase in expression of the reporter gene as repression is relieved. Hence, the signal is inverted.

Another modification of the interaction trap assay is the so-called “reverse two-hybrid system.” Under reversed two-hybrid assay conditions, as described by U.S. Pat. Nos. 5,955,280, and 5,965,368, the interaction between the fish and bait proteins results in activation of a reporter gene, which can be a negative selectable marker for cell growth. For example, the presence of the URA3 gene in the yeast may be lethal to the host cell when the host is growing on a media with 5′-FOA, a chemical that can be metabolized by the URA3 gene product into a toxic agent which kills the host cell. Thus, under such growth conditions, only when the reporter gene is inactive can the host cell survive. This system can be used to efficiently select for agents that disrupts fish-bait interaction. For example, BAK can be expressed as a bait and a “target polypeptide” that can interact with BAK, such as M11L or Bcl-2, can be expressed as a fish and used in such a reverse two-hybrid assay. Any test agent that can disrupt the interaction between BAK and its target polypeptide can then be identified and its ability as a pro- or anti-apoptosis agent further analyzed according to the instant invention. The agent can be small chemical molecules, and the experiment can be automated in a high-throughput assay. Alternatively, the test agent can be nucleic acid libraries encoding polypeptides, which, upon transfection and expression in the host cells harboring such reverse two-hybrid system, modulates the interaction of bait and fish proteins.

In a related embodiment, given the quantitative nature of the assay (the lethal dose of selective drug concentration can be adjusted based on the strength of fish-bait interaction), any agent that can potentiate the interaction of two proteins can also be identified. For example, if a first round “lead molecule” is identified in the initial screen as a weak binding partner of BAK, subsequent rounds screens employing slightly more stringent conditions can be performed to identify test agents that can strengthen the interaction between fish and bait, thus identifying potentiators of BAK-target complex formation. Such agents might be useful in promoting naturally weak interactions between BAK and a target protein under physiological conditions, thus modulating the apoptosis pathway. Similarly, the same strategy might be useful in identifying attenuators of certain bait-fish interactions and modulates apoptosis in an opposite direction.

(iii) Anti-Apoptotic Agents

In certain embodiments of the present invention, the subject method provides further assays to identify the ability of a test agent or agent to induce apoptosis. Treatment of the cell with the test agent, or expression of the agent may all that is necessary to induce an apoptotic signal which is detected by the subject method.

In other embodiments, the assay is set up to detect test compound of test agents which inhibit apoptosis, e.g., which act at a very early stage of apoptosis. In such embodiments, the apoptotic signal can be provided to the cell by selection of the growth conditions or addition of an apoptosis inducing agent (e.g., cytokines such as TNF-alpha and exogenous stimuli such as heat, radiation and chemical agents). For example, the cell can be contacted with such neurotoxic agents as Botulinum toxin type A (Botox), clomiphene, cisplatin, etoposide, teniposides, DNA alkylating agents, macromolecular synthesis inhibitors, and the like.

B. Exemplary Disorders to be Treated by Compounds or Agents Identified by Subject Methods

As set out herein, agents which are identified by the subject method as involved in the regulation of apoptosis, whether as inducer or repressor of that cellular phenomena, may have application to further development of therapeutic agents and methods, diagnostic kits and methods, and drug screening assays. The agent itself may be the therapeutic agent. For example, a small molecule, protein or antisense molecule which has been identified as an inducer of apoptosis in virally-infected cells can itself be administered in an appropriate form for uptake by such cells (e.g., in a liposomal preparation), or a gene therapy approach can be used to where the agent is a protein.

Where the agent corresponds to a native gene, the present application specifically contemplates the further development of drug screening assays which target that gene for development of agents, especially small organic molecules, which inhibit or potentiate, as appropriate, the activity of the gene/gene product. To illustrate, where the present method identified a gene which induces apoptosis of neuronal cells, the subject invention contemplates the generation of an assay which utilizes the native gene corresponding to the agent to develop inhibitors of that gene product's role in apoptosis. In another illustrative embodiment, the subject method can be used to identify genes which inhibit apoptosis of cancer cells during treatment with chemotherapeutic agents. The identified genes can then be targeted for development of small molecule inhibitors of the protective activity, and such small molecule inhibitors can be used to increase the sensitivity of the cancer cells to chemotherapeutic treatment.

Likewise, where the agent corresponds to a native gene, the level of expression or mutation of that gene can be determined in a diagnostic assay, e.g., in order to assess the risk of a certain tissue undergoing unwanted apoptosis, or to monitor the effectiveness of an apoptotic agent. That is, the present method can be used to identify molecular markers for apoptotic cells, as well as for resistance to apoptosis. The subject method now offers the means to analyze the results of certain treatments and/or compounds on apoptotic cells and also on the formation of apoptotic cells. It now becomes possible to follow the effects of the treatment and/or of compounds on the activation or inactivation of the pathway leading to apoptotic cells. It also makes a simple separation of apoptotic cells from non-apoptotic cells in a certain population possible without having to destroy the cells. The subsequently separated populations of apoptotic and non-apoptotic cells can then therefore also be used for further tests and/or clinical applications like transplantation of autologous or heterologous stem cells or bone marrow cells. It is also possible to analyze and isolate on an individual cell basis. The method according to the invention can be used on a sample of suspended cells or tissue sections.

According to the present invention, there is provided a method for identifying an agent which can be used to selectively increase the efficacy of chemotherapeutic agents against transformed cells, e.g., as part of a treatment regimen for tumors, by inhibiting bypass of apoptotic pathways in those cells. Cancer chemotherapy often kills cells by induction of apoptosis. The ability to bypass the apoptosis pathway is a common phenomena which arises in certain cancers, and can contribute to failure of current cancer chemotherapy. Thus, a compound which can selectively promote apoptosis in cancer cells could be used in the therapeutic management/treatment thereof.

Such indications include but are not limited to HIV infection, autoimmune diseases, cardiomyopathies, neuronal disorders, hepatitis and other liver diseases, osteoporosis, and shock syndromes, including, but not limited to, septicemia.

In certain embodiments, the subject method provides agents able to selectively kill cells undergoing unwanted proliferation. For instance, the subject method can be used with agents which selectively kill transformed cells, e.g., for use in cancer therapy. In other embodiments, the subject method utilizes agents which selectively kill smooth muscle cells involved in neointima formation, e.g., for the treatment of restenosis and the like. Other proliferative disorders for which the subject method can be used include psorasis and other unwanted proliferation of untransformed cells, e.g., normal diploid cells.

In still another embodiment, the subject method can be used with agents which are neuroprotective, e.g., which prevent or reduce the severity of ischemic or epoxic damage to neuronal cells, e.g., which can be used to treat cerebral ischemia (such as stroke).

Studies of the retinal pigment epithelium are directed toward understanding the etiology and development of preventive strategies for age-related macular degeneration, a leading cause of vision loss in this country. It has recently been found that oxidants induce apoptosis in the human retinal pigment epithelium by activating the mitochondrial permeability transition. Studies of the mitochondrial genome indicate oxidant-induced deletions and rearrangements increase with aging in these cells. Utilizing anti-apoptotic agents identified by the present methods, the invention specifically contemplates treatments for macular degeneration.

III. Exemplification

The invention, now being generally described, will be more readily understood by reference to the following examples, which are included merely for purposes of illustration of certain aspects and embodiments of the present invention and are not intended to limit the invention.

### EXAMPLE 1.

Virus infection of a host triggers numerous cellular defensive responses designed to limit viral replication and contain the infection. To ensure continued virion production in the face of this immune response, viruses have, in turn, developed strategies to counteract cellular defenses and maintain a suitable environment for their own replication. A key innate cellular response to infection is apoptosis (1-3), and a growing number of viral antiapoptotic proteins continue to be characterized (1, 2, 4-6).

In certain cases, the roles of these viral antiapoptotic proteins have been defined. Some, including the viral Fas-associated death domain-like IL1β-converting enzyme (FLICE) inhibitory proteins (vFLIPs), encoded by many gammaherpesviruses as well as by the molluscum contagiosum poxvirus (2, 7), target the initial signal transduction events leading to apoptosis. The vFLIPs specifically disrupt signaling by the Fas/TNF family of death receptor proteins by preventing complete assembly of the death-inducing signaling complex on the cytoplasmic domains of these receptors after activation. Viruses have also devised strategies to target the caspases, the family of apoptotic proteases that play key roles as initiators and effectors of apoptotic events. Viral proteins able to counteract the caspases include p35 of baculovirus, the poxviral serpin CrmA/Spi2, and the E3-14.7K protein of adenovirus (2, 5, 8). The baculovirus inhibitors of apoptosis proteins (IAPs) are another class of proteins implicated in the regulation of caspase activation, a function that may be linked to their ability to counter the effects of the proapoptotic proteins RPR, GRIM, HID, and DOOM in insect cells (9). Other viral proteins modulate the cell death checkpoint mediated by the Bcl-2 family of apoptotic regulators. Viral proteins such as the Bcl-2 homologues encoded by the lymphotropic gamma herpes viruses, the E1B-19K protein of adenovirus, and the 5-HL protein of African swine fever virus have sequence and/or functional homology to cellular Bcl-2 proteins and are able to abrogate the activity of the proapoptotic Bcl-2 family members, in some cases by direct physical interaction (2, 5, 7).

Recent experimental findings have indicated a pivotal role for mitochondria in the “decision to die” checkpoint regulated by Bcl-2 proteins (10-13). In this context, Bcl-2 proteins are believed to regulate the mitochondrial permeability transition (PT) pore and thereby control the release of cytochrome c and other proteins from within mitochondria into the cytoplasm. Once these proteins are able to interact with cytoplasmic components, they become proapoptotic and mediate the activation of key downstream effectors such as Caspase-3. Like their cellular counterparts, certain viral Bcl-2 family members have been associated with the mitochondrial checkpoint. In particular, stable expression of the herpes virus saimiri Bcl-2 protein in Jurkat lymphocytes has been shown to prevent loss of mitochondrial membrane potential, cytochrome c release, and Caspase-3 activation after ligation of the Fas receptor (14).

Recently, an antiapoptotic human cytomegalovirus protein, vMIA, has been described that has no homology to other known proteins and localizes to mitochondria. This protein inhibits mitochondrial changes typically associated with apoptosis, such as the release of cytochrome c into the cytoplasm and, significantly, binds the adenine nucleotide carrier subunit of the permeability transition (PT) pore (15). The characterization of novel viral proteins such as vMIA demonstrates how analysis of viral protein function can provide valuable insight into normal cellular processes.

Several poxvirus-encoded proteins have been implicated in regulating apoptotic cascades, but presently most of these proteins have an undefined mechanism of action. Included among the poxvirus apoptotic modulators are several proteins encoded by myxoma virus (16, 17), a Leporipoxvirus which is the causative agent of a lethal disease, myxomatosis, in the European or laboratory rabbit (18). Myxoma virus apoptotic modulators include M-T2, M-T4, M-T5, and M11L, as revealed by the finding that expression of these proteins is required during infection of RL-5 rabbit lymphocytes to prevent apoptosis and allow efficient virus replication (19-21).

M11L is a novel myxoma virus-encoded protein that currently has no database homologues outside the poxvirus family (22). It is 166 amino acids in length and has no distinct structural motifs apart from a hydrophobic stretch of 18 amino acids near the COOH terminus that constitutes a putative transmembrane region. M11L plays an important role in the virulence of myxoma virus during host infection. This was demonstrated during characterization of a myxoma virus variant unable to express the M11L protein as a result of a targeted gene disruption. In marked contrast to the parental virus, which gives rise to the lethal symptoms of myxomatosis, the M11L deletion mutant elicited a highly attenuated, nonlethal disease phenotype in laboratory rabbits. Despite its reduced virulence, however, the lesions produced by the M11L knockout virus were unusual, and histological analysis revealed signs of vigorous inflammatory activity. The knockout virus was also shown to be impaired in its ability to replicate in primary rabbit splenocytes (23). The attributes of M11L suggested a model in which this protein could act as a virulence factor by preventing apoptosis of leukocytes during host infection, thus compromising the effectiveness of cellular protective mechanisms designed to limit viral propagation.

In this example, we demonstrate that M11L is antiapoptotic when expressed independently from other viral proteins. Significantly, M11L localizes to mitochondria, and we provide evidence that the protection afforded by M11L influences the mitochondrial checkpoint. Finally, we show that M11L is required to maintain the viability of primary rabbit monocytes infected with myxoma virus and suggest a key role for M11L linking the inhibition of apoptosis with inflammation suppression during infection.

Materials and Methods

Cells. RL-5 rabbit CD4 lymphocytes (National Institutes of Health AIDS Reagent Program) and HeLa cells (American Type Culture Collection) were maintained in RPMI medium (GIBCO BRL) supplemented with 10% fetal bovine serum (FBS; GIBCO BRL). Primary rabbit monocytes were cultured in the same medium supplemented with 20% FBS. Rat2 fibroblasts, COS-7 monkey fibroblasts, and HepG2 human hepatocellular carcinoma cells (American Type Culture Collection) were maintained in DMEM (GIBCO BRL) supplemented with 10% FBS. BGMK monkey kidney cells (obtained from Dr. S. Dales, University of Western Ontario) were maintained in DMEM supplemented with 10% newborn calf serum (GIBCO BRL). All media contained 200 U/ml penicillin and 200 μg/ml streptomycin.

Virus Infections. Recombinant viruses used in this study have been described previously. These include vMyxlac, a control myxoma virus that produces M11L, vMyxM11L⁻ which fails to produce M11L owing to a targeted gene disruption (23), and vMyxM11L^{R}, a revertant virus in which the gene disruption has been repaired (20). A vaccinia virus construct that overexpresses M11L, VVM11L (22), and a control vaccinia virus, VV601, were also employed. Other myxoma virus constructs used in this study included the M-T2 (24), M-T4 (19), and Serp-1 (25) targeted disruption mutants. Cells were infected at a multiplicity of infection (MOI) of 10 as described (19).

Plasmid Constructs. The M11L coding sequence was amplified by PCR using the primers 5′Eco (5′-GCTAGAATTCATGATGTCTCGTTTAAAGAC-3′, SEQ ID No. 2) or 5′Xho (5′-GGATCTCGAGATGATGTCTCGTTTAAAGAC-3′, SEQ ID No. 3) and 3′Sal (5′-CGTAGTCGACTAGGTCCCTCGGTACC-3′, SEQ ID No. 4), and cloned into the T-tailed vector pT7blue (Novagen). For retrovirus-directed expression of M11L, the gene was subcloned into the murine leukemia virus-based vector pBabePuro (26) to produce the vector pBabe-PuroM11L. To allow the expression of GFP-M11L, a fusion protein consisting of green fluorescent protein (GFP) appended to the NH2 terminus of M11L, the M11L coding sequence was cloned into the GFP expression vector pS65T-C1 (Clontech) encoding the S65T variant of GFP so that the M11L sequence was inserted downstream of and in frame with the GFP coding sequence. A mutated form of M11L was created using a PCR based approach. The M11L coding sequence was amplified using the 5′Xho primer detailed above and a 3′ primer M11Lstop (5′-AACTGCCGCGGTTAGATAGACGGATCATTT-3′, SEQ ID No. 5) incorporating a stop codon in place of the codon specifying isoleucine at position 143, the first amino acid of the hydrophobic region. A restriction fragment containing the mutated codon was excised from this amplified PCR product and used to replace the corresponding fragment of the wild-type M11L gene. These constructs were subcloned into the pEGFP-C1 vector (Clontech) to allow expression of the same M11L constructs appended to the COOH terminus of the EGFP (enhanced) variant of GFP, which has a higher fluorescence intensity. To identify a minimal mitochondrial targeting signal contained in M11L, a restriction fragment containing the coding sequence for the last 25 amino acids of the protein was cloned into pEGFP-C1. As a positive control, the Bcl-2 coding sequence (provided by Dr. S. Farrow, Glaxo Wellcome Research and Development, Stevenage, UK) was cloned into pEGFP-C1 to allow expression of an EGFP-Bcl-2 chimeric protein. Correct construction of all GFP chimeras was verified by DNA sequencing analysis.

Ectopic Expression of M11L. Control pBabePuro and pBabe-PuroM11L vectors were transiently transfected into BOSC 23 cells and packaged into ecotropic virus particles as described (27). After infection of Rat2 fibroblasts, pooled clones of cells that had stably incorporated the respective proviruses were selected on the basis of ability to grow in the presence of 2.5 μg/ml puromycin, and designated Rat2puro and Rat2M11L. Expression of the M11L protein by Rat2M11L cells was verified by immunoblot analysis (data not shown). Transient expression of GFP chimeric proteins for flow cytometry was accomplished by transfecting HeLa cells with GFP plasmid constructs using the Lipofectin Plus reagent (GIBCO BRL) as described (28). Analysis was conducted 24 h after transfection.

Induction and Measurement of Apoptosis. Apoptosis was induced by addition of staurosporine (Sigma Chemical Co.) to the cell culture medium at a final concentration of 2 μM. Apoptotic cells were identified by measuring the characteristic elevation in levels of nicked DNA using the previously described terminal deoxynucleotidyl transferase-mediated dUTP-fluorescein nick end labeling (TUNEL) reaction (19, 29).

Measurement of Caspase-3 Activation. Rat2puro and Rat2-M11L cells (10⁶) were incubated with 5 μM staurosporine for up to 4 h. Caspase-3 activation was detected by SDS-PAGE and immunoblot analysis as described (30) using an antibody directed against the large subunit of the active enzyme (provided by Dr. D. Nicholson, MerckFrosst Center for Therapeutic Research, Montreal, Quebec, Canada).

Confocal Microscopy. To study M11L localization in myxoma virus-infected cells, BGMK cells grown on coverslips were treated 20 h after infection with the mitochondrial-specific fluorescent marker Mitotracker Red CXMRos (Molecular Probes) at a final concentration of 30 ng/ml. Accumulation of the dye was allowed to occur for 20 min at 37° C. The cells were fixed with 2% paraformaldehyde/PBS for 30 min at room temperature and permeabilized for 2 min with cold 0.1% Triton X-100/0.1% sodium citrate buffer. Cells were then incubated for 20 min at room temperature with a polyclonal rabbit anti-M11L antibody (22) diluted 1:50 in PBS followed by incubation for 20 min at room temperature with a secondary FITC-conjugated anti-rabbit antibody (Jackson ImmunoResearch Laboratories) at a dilution of 1:200 in PBS. Coverslips were mounted using 50% PBS/50% glycerol solution, and confocal images were obtained using an LSM510 laser scanning confocal microscope mounted on a Zeiss Axiovert 100M microscope equipped with a 63×1.4 oil immersion Plan-Apochromat objective. FITC excitation was induced by illumination at 488 nm, and the fluorescent signal was collected using a 505-530-nm band pass filter. Mitotracker Red fluorescence was induced by illumination at 543 nm and was detected using a 560-nm long pass filter. Images of live cells were obtained by growing cells in 3.5 cm diameter cell culture dishes modified so that a section of the base was replaced by a glass No. 1 coverslip (Fisher Scientific). Partially confluent monolayers of COS-7 or HeLa cells were each transfected with 4 μg of DNA using the Lipofectin reagent and OptiMEM medium (GIBCO BRL) according to the manufacturer's specifications. The transfected cells were incubated for 24 h, and mitochondria were stained with Mitotracker Red CXMRos (Molecular Probes) by addition of the dye to the culture medium at a concentration of 15 ng/ml. Cells were replenished with RPMI medium lacking phenol red (GIBCO BRL) and examined by confocal microscopy using the same filter settings described above. Red and green signals were collected sequentially to eliminate bleed-through. Neutral density filters were set at levels of 80% or higher to minimize photobleaching.

Analysis of Protease Sensitivity. HepG2 cells (5×10⁵) were infected at an MOI of 10 with the M11L-overexpressing vaccinia virus VVM11L, or with the control virus VV601 for 12 h. The cells were harvested and resuspended in 200 μl digitonin lysis buffer (75 mM NaCl, 1 mM NaH₂PO₄, 8 mM Na₂HPO₄, 250 mM sucrose, and 95 μg/ml digitonin) at 4° C. for 5 min to permit selective permeabilization of the plasma membrane without disrupting intracellular membranes (31). Both samples were divided into four aliquots of 50 μl each and centrifuged at 15,000 g for 15 min at 4° C., and the supernatants were retained. The pellets were resuspended in 50 μl digitonin lysis buffer. Duplicate samples were then treated with the protease inhibitor PMSF (Sigma Chemical Co.) at a final concentration of 1 mM immediately or treated with 2.5 mg/ml proteinase K (Boehringer Mannheim) at 4° C. for 20 min before the reaction was quenched by PMSF addition. One of the duplicate samples was then resuspended in SDS sample buffer, and the whole cell lysate was used to detect cytochrome c oxidase subunit IV (COX IV). The other sample was used to immunoprecipitate M11L. This was achieved by dilution of the sample to 1 ml in NP-40 lysis buffer (50 mM TrisHCl, pH 8, 150 mM NaCl, 1% NP-40, 0.5% sodium deoxycholate, 2 mM EDTA, and 1 mM PMSF) and agitation at 4° C. for 30 min. Insoluble material was pelleted at 4° C. by centrifugation at 15,000 g for 10 min. The supernatant was removed, and 10 μl of rabbit polyclonal antibody (22) was added. The samples were incubated at 4° C. with constant agitation for 2 h, and 20 μl of a 50 % slurry of protein A-Sepharose beads (Amersham Pharmacia Biotech) was added before further incubation for 1 h. The beads were then pelleted by low speed centrifugation, washed in lysis buffer, and boiled in SDS sample buffer for 5 min. For COX IV and M11L detection, proteins were separated by SDS-PAGE on a 15% gel and electroblotted onto Immobilon-P membrane (Millipore). COX IV was detected using a primary mouse mAb (Molecular Probes) at a concentration of 0.4 μg/ml and a secondary anti-mouse horseradish peroxidase conjugate (BioRad) at 1:6,000 dilution followed by enhanced chemiluminescence detection (ECL; Amersham Pharmacia Biotech). M11L was detected using the primary rabbit polyclonal antibody at a dilution of 1:500 and a secondary protein A-horseradish peroxidase conjugate (Pierce Chemical Co.) at a dilution of 1:10,000 followed by ECL detection.

Measurement of Mitochondrial Membrane Potential in Cells Expressing M11L Constructs. Rat2puro or Rat2M11L cells were cultured in 12-well plates (5×10⁵ cells/well) and treated with 2 μM staurosporine for 4 h. As the staurosporine stock was prepared in DMSO, control cells were treated with an equivalent amount of DMSO alone. Thereafter, the cells were stained with the green fluorescent dye 3,3′dihexyloxacarbocyanine iodide (DiOC₆; Molecular Probes). The fluorescence intensity of this dye has been found to correlate with the mitochondrial membrane potential and provide an accurate measure of loss of this potential during apoptosis (32). Cells were incubated with 0.5 nM DiOC₆ at 37° C. for 10 min, harvested with trypsin, and then washed and resuspended in RPMI lacking phenol red (GIBCO BRL). To verify that a decrease in the fluorescence signal intensity did accompany a loss of mitochondrial membrane potential, control cells were treated with the protonophore carbonyl cyanide mchlorophenyl hydrazone (CCCP; Molecular Probes), which causes dissipation of the proton gradient across the mitochondria inner membrane (32). CCCP was added to the cell culture medium at a final concentration of 50 μM during and after dye addition. Flow cytometric analysis was conducted using a Becton Dickinson FACSCalibur™ flow cytometer equipped with an argon ion laser with 15 mW of excitation at 488 nm. Data were acquired at 10,000 cells per sample, and the fluorescent signal due to excitation of DiOC₆ at 488 nm was detected through the FL1 channel equipped with a 530 nm filter (30 nm band pass). Light scatter signals were acquired at linear gain, and fluorescence signals were acquired at logarithmic gain. HeLa cells transiently expressing GFP plasmid constructs were treated with 2.5 μM staurosporine, and control samples were treated with DMSO alone. Measurement of changes in mitochondrial membrane potential in this system was conducted using the ethyl ester of tetramethylrhodamine (TMRE; Molecular Probes), as the accumulation of this dye in the mitochondria of HeLa cells correlates directly with the magnitude of the negative mitochondrial membrane potential. In addition, this dye displays no appreciable nonspecific binding, selfquenching, or cellular toxicity (33, 34). TMRE was added to cells at a final concentration of 0.1 μM, and CCCP was added to a control sample. Flow cytometric analysis to detect green fluorescence due to GFP and orange fluorescence due to TMRE was conducted using a FACSCalibur™ instrument specified above. GFP fluorescence was collected after a 530/30-nm band pass filter, and TMRE fluorescence was acquired through a 585/42-nm band pass filter. Electronic fluorescence compensation was set to eliminate any spectral overlap of the emitted signals. Data were acquired at 10,000 cells per sample with fluorescence signals at logarithmic gain. The percentage of GFP-positive cells that were also TMRE-positive was then calculated for each gated cell population.

Isolation, Infection, and Analysis of Primary Rabbit Monocytes. For each experiment, heparinized blood was obtained by cardiac puncture from a healthy New Zealand White laboratory rabbit and subjected to Ficoll-Paque (Amersham Pharmacia Biotech) density gradient separation. The buffy coat containing white blood cells was collected, and cells were cultured in RPMI medium containing 20% FBS for 4 h; the monocyte population was enriched as a result of adherence to the plastic culture dish. The adherent cells were detached from the plastic support using warm 1×SSC (150 mM sodium chloride, 15 mM sodium citrate, pH 7.5) and infected with appropriate viruses at an MOI of 10. Typically, infection rates exceeded 70% as verified by staining with X-gal (23). The infected cells were cultured in medium containing 20% FBS for 12 h, fixed in 2% paraformaldehyde/PBS, and apoptotic cells were detected using the TUNEL reaction. The monocyte population in each sample was identified by indirect immunofluorescence by incubation with a mouse anti-rabbit CD11b antibody (Spring Valley) at a dilution of 1:50 at room temperature for 20 min, followed by incubation with a PE-conjugated F(ab′)₂ goat anti-mouse secondary antibody (Dako) at a dilution of 1:20 at room temperature for 20 min. Data were acquired using a Becton Dickinson FACScan™ flow cytometer using the same settings described for the FACSCalibur™ instrument. The fluorescein-dUTP signal from TUNEL-positive cells and PE fluorescence from CD11b-positive cells was analyzed for the gated cell population.

Results

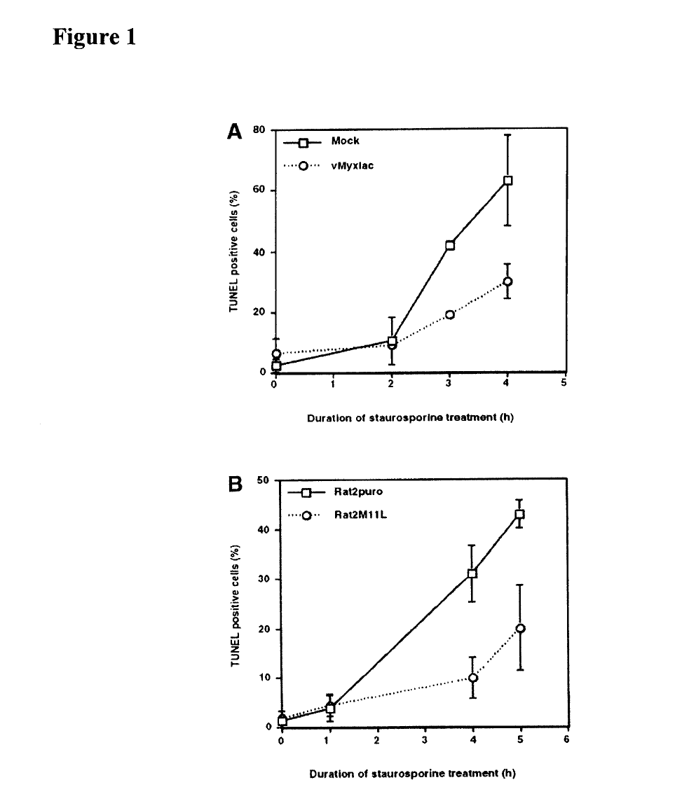

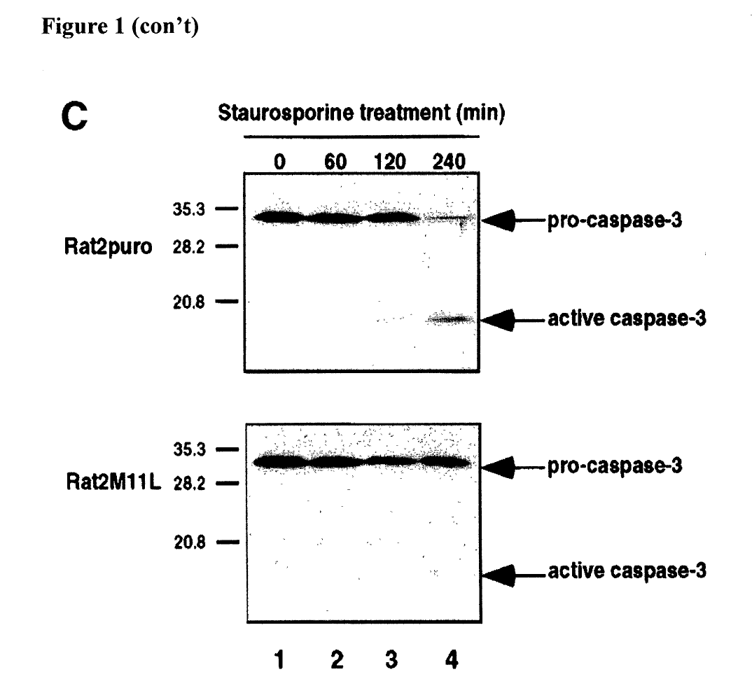

The M11L Protein of Myxoma Virus Is Antiapoptotic. In view of the experimental findings that several different targeted gene disruptions in the myxoma virus genome yielded virus variants with proapoptotic phenotypes (19-21), it followed that myxoma virus encodes more than one protein able to directly modulate apoptotic cascades. First, however, it was of interest to ascertain whether myxoma virus infection provided cells with protection from exogenous apoptotic stimuli in addition to the process of virus infection itself. RL-5 rabbit T lymphocytes were mock-infected or infected with myxoma virus (vMyxlac), and 12 h after infection were treated with the apoptosis inducer staurosporine at a concentration of 2 μM for up to 4 h. Apoptotic cells were identified by measuring the characteristic elevation in the levels of nicked DNA using TUNEL analysis. After staurosporine treatment, the percentage of TUNEL positive (apoptotic) cells was found to be substantially higher in the mock infected cell population than was the case with infected cells (FIG. 1A). Thus, myxoma virus infection counteracts the proapoptotic effects of staurosporine.

In contrast to parental myxoma virus, the M11L knockout virus has a distinct proapoptotic phenotype when infecting RL-5 cells (20). This finding suggested that, during the normal course of myxoma virus infection, M11L is one of the factors that provides an antiapoptotic function (16).

Therefore, we investigated the possibility that M11L has protective, antiapoptotic properties that are able to extend beyond the context of virus infection. To achieve this, we expressed M11L in Rat2 fibroblasts using a retrovirus based approach. The ability of staurosporine to induce apoptosis in M11L-expressing Rat2M11L cells and control Rat2puro cells, transfected with empty vector alone, was monitored by measuring DNA fragmentation using the TUNEL assay.

Whereas 2 μM staurosporine treatment over a 5 h duration produced a steady increase in the levels of TUNEL-positive Rat2puro cells, the levels of TUNEL-positive Rat2M11L cells were considerably reduced under the same conditions, indicating that M11L expression alone confers resistance to staurosporine-induced apoptosis (FIG. 1B). This result provided the first evidence that M11L is antiapoptotic when expressed independently of other myxoma virus proteins and is able to function in cells derived from a species other than rabbit.

To confirm the idea that M11L directly impacts an apoptotic cascade, we asked whether M11L interferes with end-stage apoptotic processes, such as the cleavage and activation of the effector caspase, Caspase-3. To address this question, we monitored Caspase-3 processing in Rat2puro and Rat2M11L cells by immunoblot analysis using an antibody directed against the large subunit of the active caspase.

Treatment of Rat2puro cells with 5 μM staurosporine over a duration of 4 h resulted in activation of Caspase-3 as revealed by reduction in the amount of the 32 kD proenzyme and appearance of the p19 cleavage product, a component of the active enzyme (FIG. 1C, top). In contrast, Caspase-3 activation in Rat2M11L cells after the same treatment was substantially reduced (FIG. 1C, bottom), with only a low level of activation being apparent after 4 h treatment (compare lane 4, top and bottom). In additional experiments (data not shown), M11L expression in Rat2 fibroblasts was found to inhibit staurosporine induced cleavage of poly(ADPribose) polymerase, a substrate of effector caspases such as Caspase-3 (35). These findings suggest that M11L impedes apoptotic signaling events upstream of Caspase-3 activation.

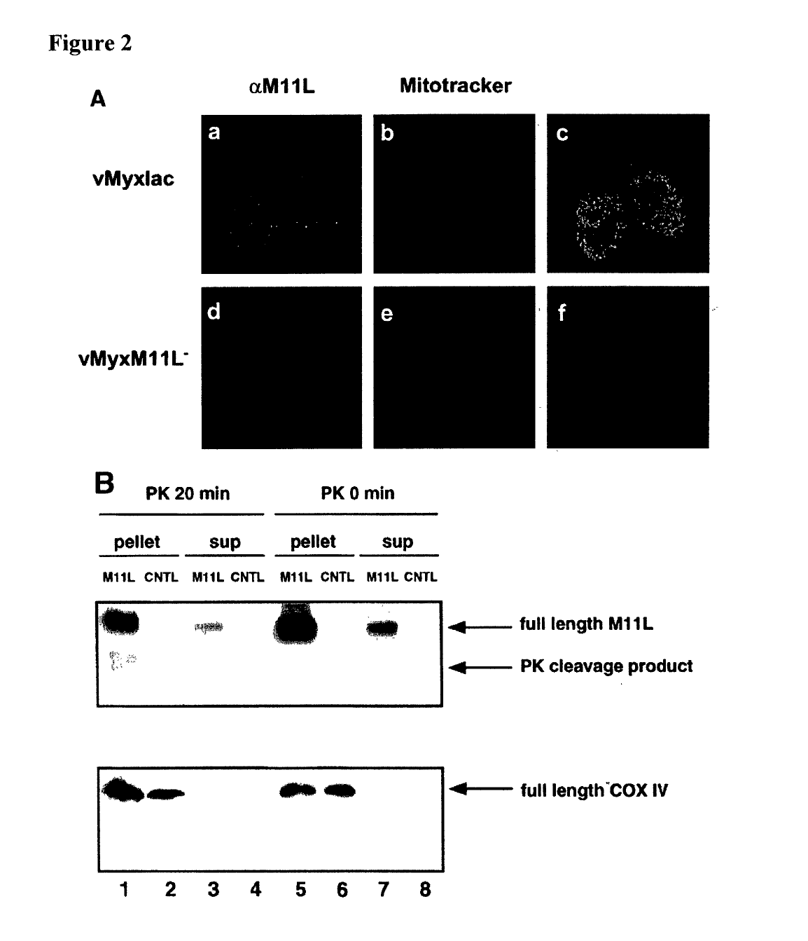

M11L Localizes to Mitochondria in Infected Cells. Our studies demonstrated that M11L is antiapoptotic, but database searches failed to identify similarities between M11L and any other protein of known function. However, sequence analysis of M11L revealed the presence of a COOH terminal putative transmembrane domain. This observation prompted us to investigate the subcellular localization of M11L during myxoma virus infection. BGMK cells were infected with M11L-expressing myxoma virus (vMyxlac) or the M11L knockout virus (vMyxM11L⁻), and 20 h after infection, M11L was visualized by indirect immunofluorescence and confocal microscopy. As expected, M11L was detected in vMyxlac-infected cells (FIG. 2A, panel a) and not in cells infected by the knockout virus (FIG. 2A, panel d). In addition, M11L was observed to have a punctate, cytoplasmic distribution reminiscent of mitochondrial targeting. To determine whether M11L did, indeed, localize to mitochondria, cells were treated with the mitochondrion specific dye Mitotracker Red (FIG. 2A, panels b and e). When the fluorescent signals due to M11L and Mitotracker Red were superimposed, a uniform yellow image was produced, indicating that the two signals were coincident (FIG. 2A, panel c). No such signal was observed in the case of cells infected with the knockout virus (FIG. 2A, panel f). This result provides evidence that, within myxoma virus-infected cells, intracellular M11L localizes predominantly to mitochondria.

To investigate the topology of M11L within mitochondria, we conducted experiments to determine whether M11L is proteinase K sensitive. For these experiments, we used the human hepatocyte-derived HepG2 cell line, as these cells contain a high proportion of mitochondria. Digitonin extracts of HepG2 cells were separated into pellet and supernatant fractions after infection for 12 h with the M11L overexpressing virus vector VVM11L or the control virus VV601 (which does not express M11L). Samples were treated with proteinase K or left untreated before immunoblot analysis of M11L or COX IV, a protein that resides in the inner mitochondrial membrane. As can be seen from FIG. 2B (top), the majority of the M11L protein is present in the pellet fraction (lanes 1 and 5), which contains membrane-associated components. A small proportion of M11L can be detected in the soluble fraction (lanes 3 and 7), as would be expected for a protein produced by a cytoplasmic virus. M11L protein was not present in cells infected with the control virus, as expected (lanes 2, 4, 6, and 8). COX IV was only detected in the pellet fractions (FIG. 2B, bottom, lanes 1, 2, 5, and 6). However, when the protease sensitivity of M11L was assessed, it was evident that M11L present in the pellet fraction was proteinase K sensitive (FIG. 2B, top, lane 1). M11L present in the supernatant fraction was also protease sensitive, as revealed after overexposure of the immunoblot (data not shown). In contrast, COX IV, present in the pellet fraction, was protease resistant (FIG. 2B, bottom, lanes 1 and 2). These data provide evidence that M11L, although associated with mitochondria, is exposed on the cytoplasmic face of the organelle.

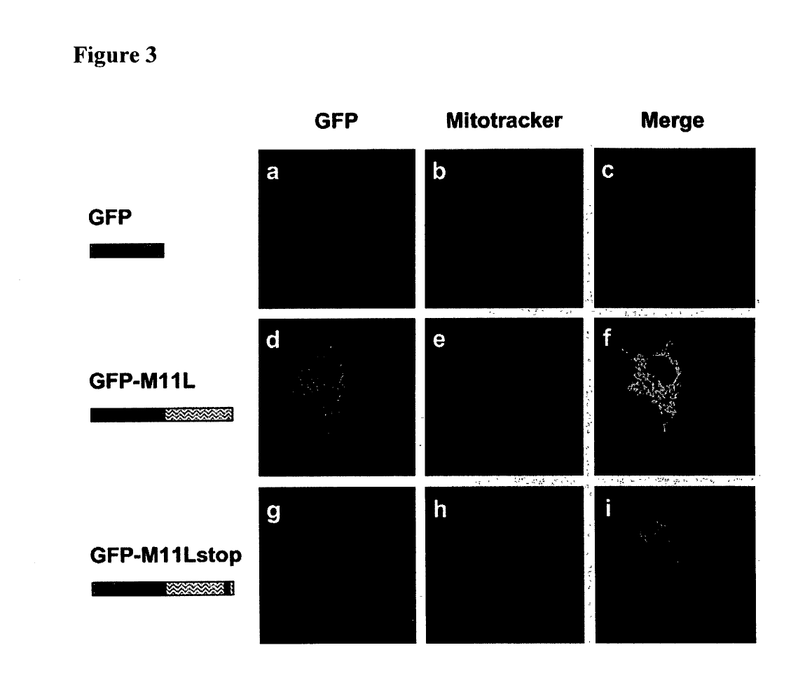

M11L Expressed in Uninfected Cells Localizes to Mitochondria and Contains a COOH terminal Mitochondrial Targeting Signal. We next sought to determine the spatial distribution of M11L in live, transfected cells. A chimeric form of M11L bearing an NH2 terminal GFP tag was expressed in both COS-7 and HeLa cells. As was the case for M11L detected in virus infected BGMK cells, GFP tagged M11L displayed a punctate cytoplasmic distribution both in COS-7 cells (FIG. 3d) and HeLa cells (data not shown) which was suggestive of association with intracellular membranes. Upon comparison of the GFP-M11L signal fluorescence pattern with that of Mitotracker Red in the same cells (FIG. 3e), the two signals were found to be coincident (FIG. 3f), indicating that GFP-M11L localizes to mitochondria. In contrast, GFP alone produced a diffuse signal throughout COS-7 cells (FIGS. 3, a and c), reflecting its lack of intracellular targeting and lack of correspondence with Mitotracker Red staining (FIG. 3b). These experiments therefore show that a GFP tag, when added to the NH2 terminus of M11L, does not alter the mitochondrial localization of the protein, and that this localization is consistent in cells having different species of origin.

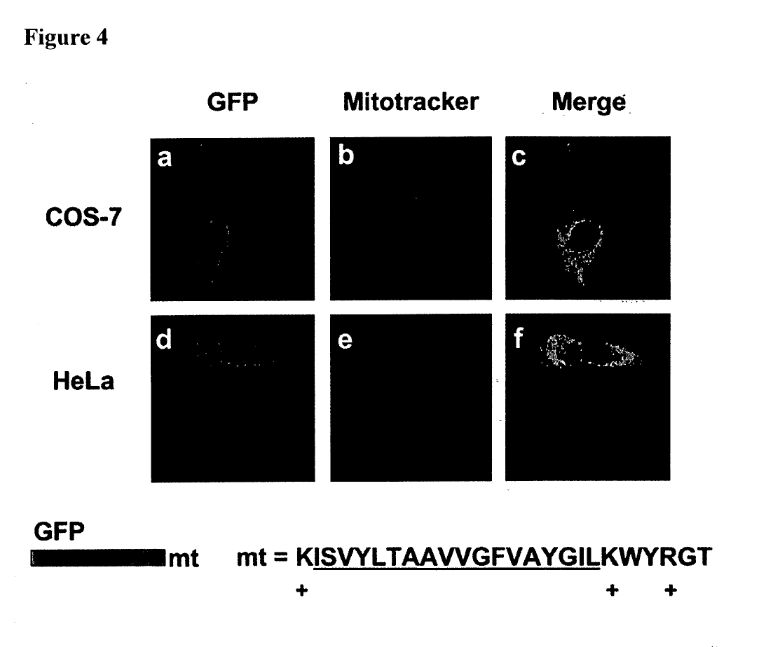

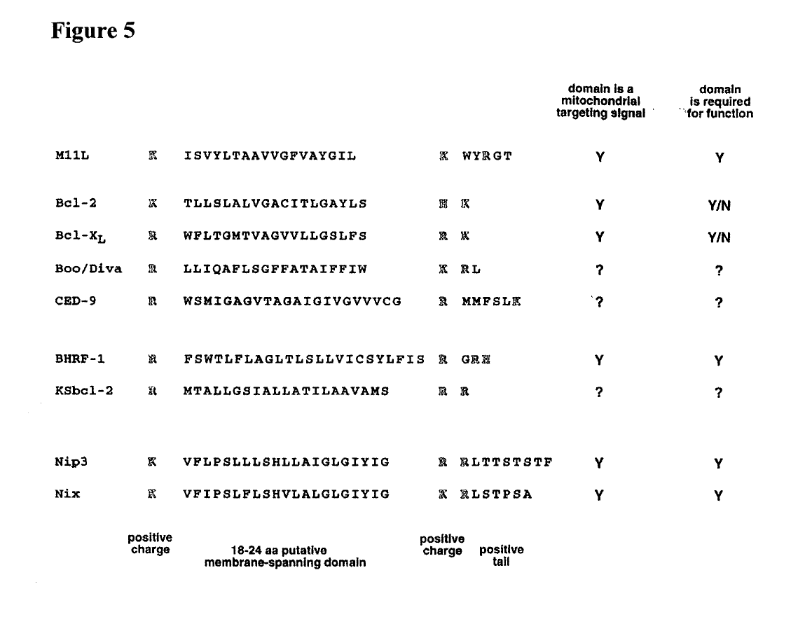

Since the COOH terminal 24 amino acids of M11L include a stretch of 18 amino acids that constitute a putative transmembrane domain, we wished to determine whether this domain was important for targeting. To address this question, the localization of a GFP-tagged, truncated form of M11L lacking the COOH terminal 24 amino acids was investigated. Interestingly, this truncated form of M11L was distributed diffusely throughout the cytoplasm and nucleus in COS-7 cells (FIGS. 3, g and i) and showed no correspondence with the staining pattern due to Mitotracker Red (FIG. 3h). The same result was obtained using HeLa cells (data not shown). This shows that the COOH terminal 24 amino acids are necessary for M11L targeting to mitochondria, and removal of this domain, which includes the putative transmembrane region and short 6-amino acid positively charged tail, prevents mitochondrial localization. We next sought to determine whether the 25-amino acid COOH terminal region of M11L alone was sufficient for mitochondrial targeting. The coding sequence for this minimal region (designated mt, FIG. 4) was appended to GFP and was found to direct GFP to punctate cytoplasmic structures in both COS-7 (FIG. 4a) and HeLa (FIG. 4d) cells. When the distribution of this fluorescent signal was compared with that of Mitotracker Red (FIGS. 4, b and e) in the same cells, the two signals were found to be coincident (FIGS. 4, c and f). Hence, the COOH terminal 25 amino acids of M11L comprise a signal that is sufficient for mitochondrial targeting. This targeting motif includes an 18-amino acid putative transmembrane domain flanked by positively charged lysine residues adjacent to a short 6-amino acid COOH terminal tail with a net positive charge (FIG. 5). A truncated form of this sequence consisting of only the last 19 amino acids of M11L failed to localize GFP to mitochondria (data not shown), indicating the requirement for a hydrophobic stretch sufficiently long to form a transmembrane segment within the targeting signal. The M11L COOH terminal targeting signal conforms to a newly described consensus for directing proteins to mitochondria. This consensus consists of a hydrophobic region flanked by positively charged residues adjacent to a short, positively charged tail and was initially identified in vesicle associated membrane protein 1B (VAMP1B), monoamine oxidase A and B, and Bcl-2 (36). We found this consensus to be additionally present in diverse Bcl-2 family members (FIG. 5).

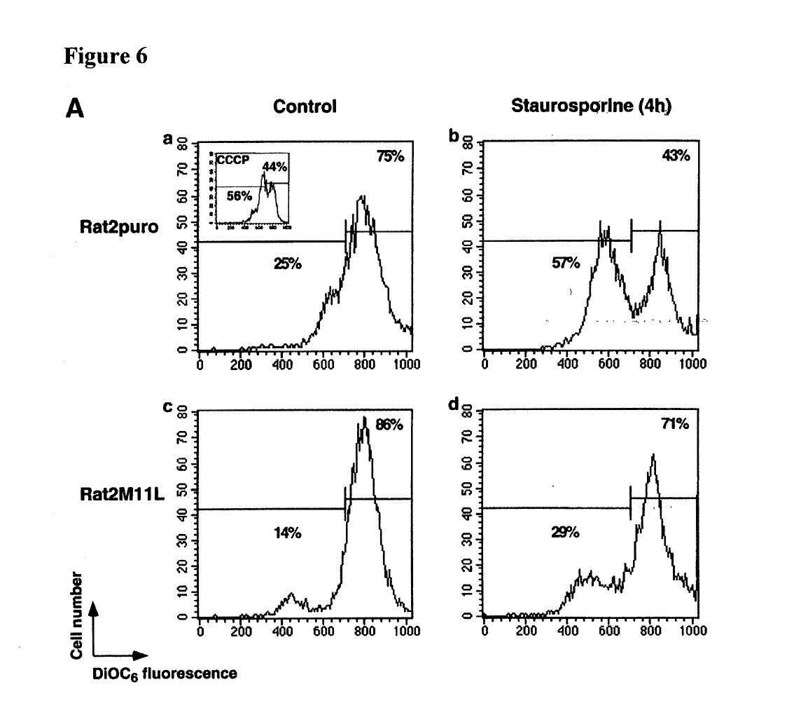

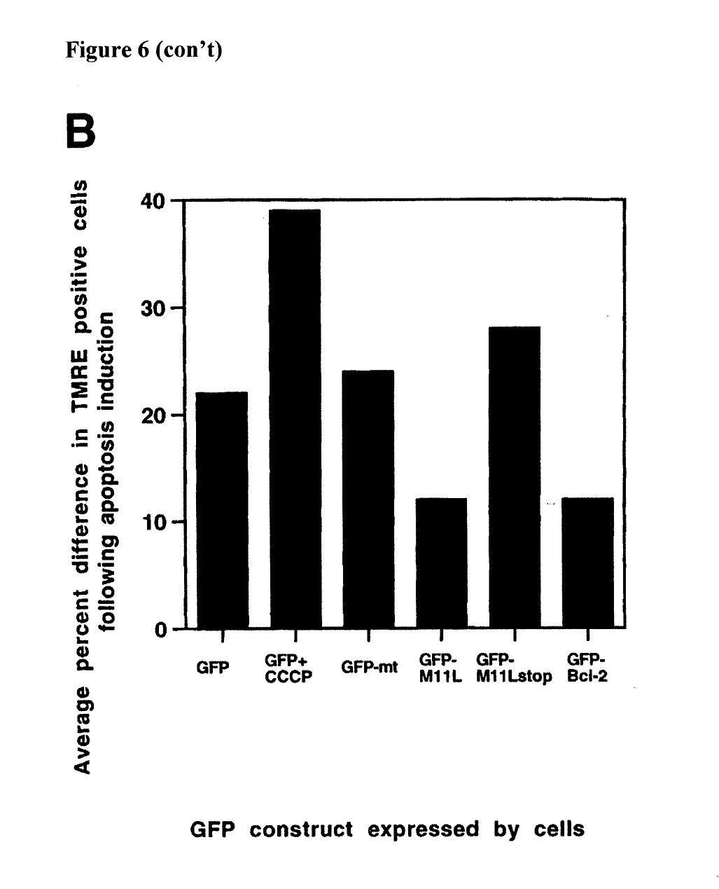

M11L Prevents Mitochondria from Undergoing a Permeability Transition after Apoptosis Induction. Since M11L inhibits apoptosis and localizes to mitochondria, it was of interest to determine whether M11L could function by protecting mitochondria from apoptotic changes, notably a permeability transition revealed by loss of electrical potential difference across the inner membrane. The mitochondrial membrane potential in Rat2puro and Rat2M11L cells was measured as a function of DiOC₆ fluorescence in the presence or absence of staurosporine treatment. Representative results from one of three experiments shown in FIG. 6A reveal that the mitochondria of control cells from both cell lines stained brightly (FIG. 6A, panels a and c). Fluorescence was diminished in the presence of the protonophore CCCP, an uncoupler of the electron transport chain that induces collapse of the mitochondrial inner membrane potential (FIG. 6A, panel a insert). However, when subjected to the proapoptotic effects of staurosporine, mitochondria in Rat2puro cells were found to be considerably more sensitive to loss of membrane potential than were Rat2M11L cells (FIG. 6A, panels b and d). These results indicate that M11L can protect mitochondria from undergoing a permeability transition after receipt of an apoptotic signal induced by staurosporine. Similarly, we investigated the ability of transiently expressed GFP constructs to protect HeLa cells from loss of mitochondrial membrane potential after staurosporine treatment. TMRE was used to detect mitochondrial changes in this series of experiments because, like DiOC₆, it displays decreased fluorescence intensity as mitochondrial membrane potential diminishes but it emits a signal in the orange spectral range and therefore does not interfere with GFP fluorescence. TMRE has been successfully used to monitor mitochondrial function in HeLa cells by confocal microscopy (33) and, in this study, was also found to be suitable for flow cytometric analysis, as is the related methyl ester TMRM (32). The percentage of cells expressing GFP constructs that were also TMRE-positive was determined in control and staurosporine treated cells. The reduction in the percentage of TMRE-positive cells after staurosporine treatment was then calculated. The average results for two separate experiments are graphically represented in FIG. 6B. As shown by this figure, expression of GFP alone, GFP targeted to mitochondria (GFP-mt), and the nontargeted GFP-M11Lstop truncation mutant all failed to prevent a loss of TMRE fluorescence. This suggests that these constructs cannot protect mitochondria from undergoing a loss of membrane potential after staurosporine treatment. Treatment of GFP expressing cells with CCCP also induced a marked loss of TMRE fluorescence. In contrast, expression of the GFP-M11L chimera and the positive control GFP-Bcl-2 construct resulted in TMRE fluorescence being retained in HeLa cells after the same treatment. This indicates that M11L, like Bcl-2, can protect mitochondria in HeLa cells from undergoing a permeability transition induced by the proapoptotic effects of staurosporine. This property is dependent on M11L being correctly localized to mitochondria.

M11L Is Required to Prevent Apoptosis during Infection of Primary Rabbit Monocytes. On the basis of the experimental findings described here, it would be predicted that infection of rabbit leukocytes by the M11L knockout virus would result in elevated levels of apoptosis in these cells. Apoptosis is normally an immunologically silent event (37), and therefore the strongly proinflammatory disease phenotype elicited in rabbits infected with the knockout virus is a seemingly conflicting observation. However, it has been reported that monocyte apoptosis has the unusual property of promoting inflammation (37, 38). Therefore, we decided to test whether infection of monocytes with the M11L knockout virus was able to induce apoptosis in these cells, a situation that could explain the proinflammatory phenotype of this virus.

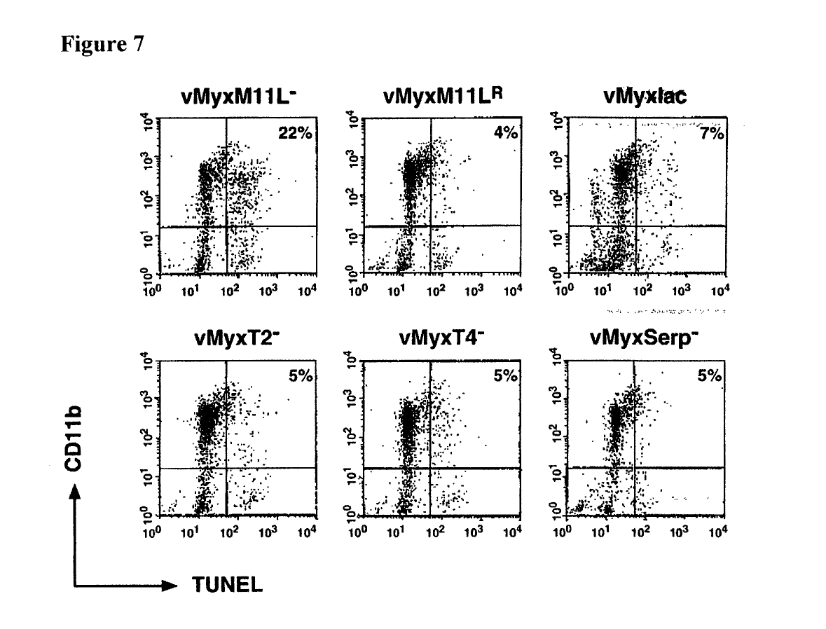

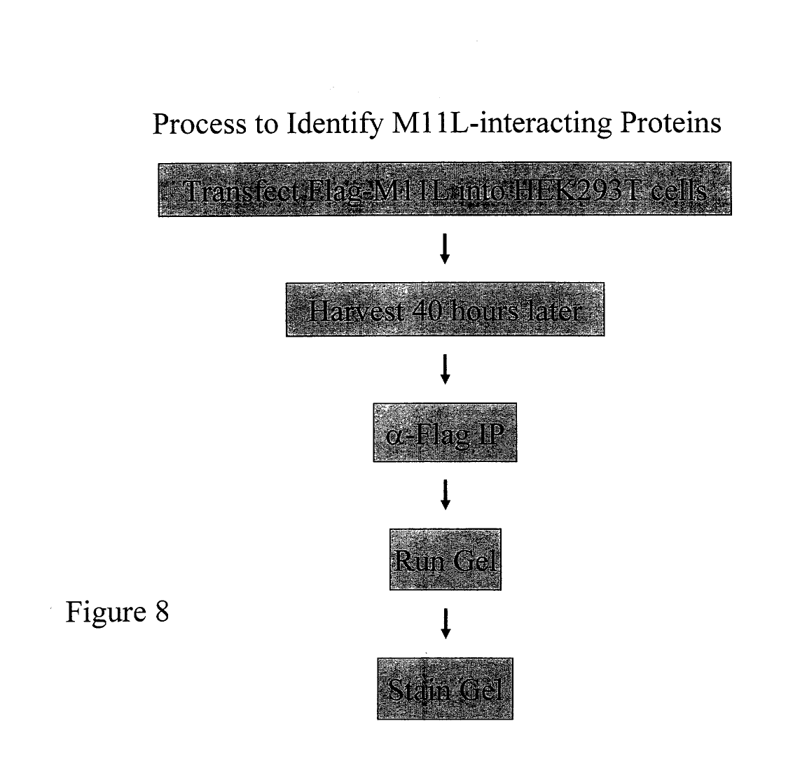

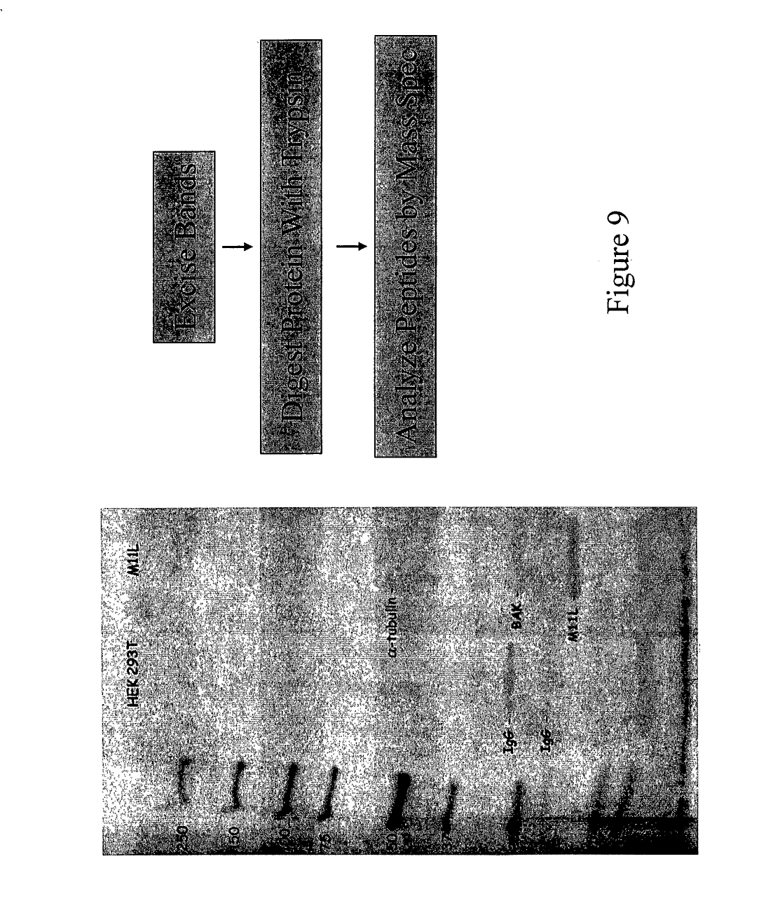

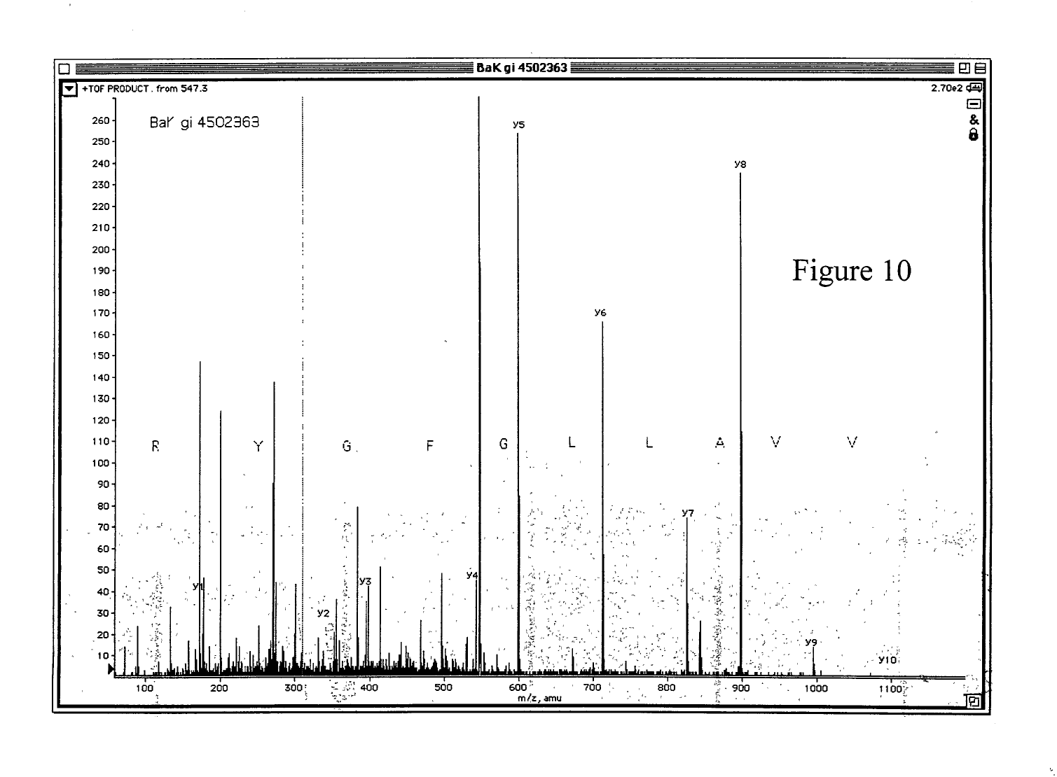

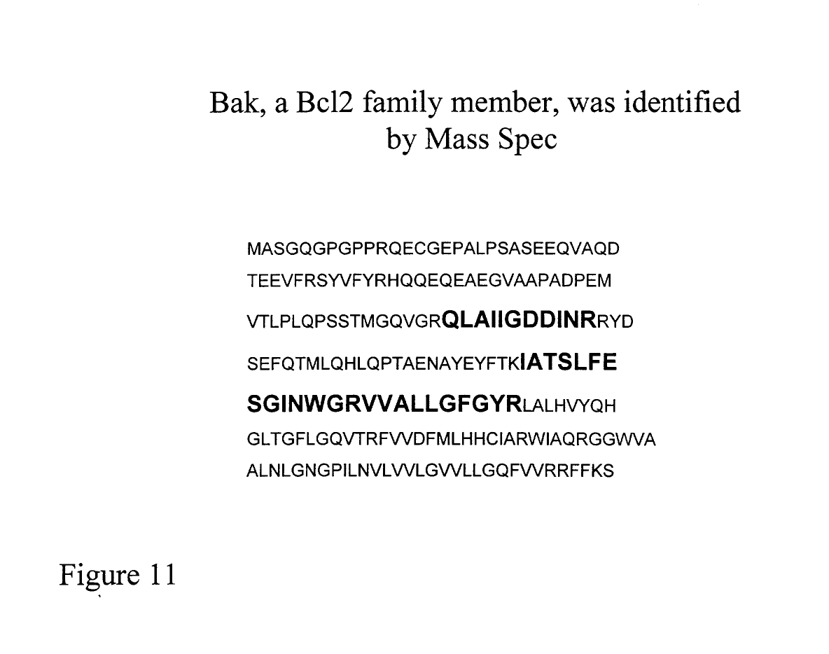

Primary rabbit monocytes were isolated from peripheral rabbit blood and infected with several myxoma virus constructs. After 12 h of infection, apoptotic cells were detected by means of the TUNEL reaction, and cells of the monocyte lineage were identified by the presence of the CD11b surface marker. Populations of dual positive cells representing apoptotic monocytes were quantitated by flow cytometry. FIG. 7 shows representative results from one of four separate experiments and reveals that only the M11L knockout virus induced notable levels of apoptosis in primary rabbit monocytes. In contrast, when monocytes were infected with virus expressing M11L, including the control vMyxlac virus, the M11L revertant virus, and myxoma virus constructs with other targeted gene disruptions, no elevation in levels of apoptosis was observed. This finding is of interest, as the M11L, M-T2, and M-T4 proteins are all required to prevent apoptosis during infection of the RL-5 lymphocytes, and disruption of any one of these genes produces a virus with a proapoptotic phenotype in this cell line. In contrast, there appears to be a unique requirement for M11L production during primary rabbit monocyte infection. Virus constructs unable to produce either the M-T2 or M-T4 protein, but still able to produce M11L, do not have a proapoptotic phenotype in primary monocytes. This result identifies a cell lineage where M11L production is functionally significant in preventing apoptosis during infection, and provides an explanation for the seemingly conflicting observations that the M11L knockout virus induces apoptosis but also produces lesions with extensive infiltrations of inflammatory cells in infected animals.

Discussion

The unique role of M11L in the virulence of myxoma virus was indicated by two previous experimental observations. First, in contrast to control virus, an M11L knockout myxoma virus elicited a markedly attenuated disease phenotype associated with unusual tumor-like lesions containing large numbers of infiltrating inflammatory cells (23).

Second, M11L was identified as a factor required to prevent apoptosis during myxoma virus infection of RL-5 lymphocytes in vitro (20). These observations suggested a model in which M11L acts as a virulence factor by virtue of its ability to prevent infected leukocytes from initiating a protective apoptotic response, thereby promoting viral replication. These results suggested that M11L exerts a host cell-protective effect when the process of infection itself serves as the apoptotic trigger. Here, we show for the first time that M11L, when expressed independently from other viral proteins, can protect these cells from the proapoptotic effects of another inducer, namely staurosporine. The finding that M11L can prevent Caspase-3 activation and poly(ADPribose) polymerase cleavage suggests that this protein has a direct effect on a strategic step in an apoptotic cascade upstream of Caspase-3.

In this study, we investigated the intracellular localization of M11L and demonstrated that this protein is primarily targeted to mitochondria. A previous study (22) based on indirect immunofluorescence analysis of nonpermeabilized cells revealed that M11L can be detected on the surface of infected cells. However, we have shown that surface M11L probably represents only a minor proportion of the total amount of protein produced in myxoma virus-infected cells. The current study used the wild-type protein expressed in the context of myxoma virus infection as well as a plasmid-encoded GFP-M11L chimera visualized in live, transfected cells to investigate the protein's intracellular localization. We demonstrated that the majority of the M11L protein within cells is targeted to mitochondria and remains accessible to proteinase K digestion. This indicates that M11L is associated with the outer mitochondrial membrane and is oriented towards the cytoplasm. Hence, M11L has an intracellular distribution similar to many Bcl-2-like antiapoptotic proteins of both cellular and viral origin (11, 13). M11L contains a signal within the COOH terminal 25 amino acids that is necessary and sufficient for mitochondrial targeting. This mitochondrial COOH terminal targeting signal conforms to a newly proposed consensus domain which takes the form of a hydrophobic region flanked by positively charged residues adjacent to a positively charged tail (36). Interestingly, COOH terminal targeting motifs responsible for directing other proteins involved in apoptosis to the outer mitochondrial membrane are also found to conform to this consensus (FIG. 5). Included in this category are Bcl-2 (31, 39), BClX_{L} (40), BHRF1 (41), Nip3 (41, 42), and Nix (43). This motif is also present in the Bcl-2 family members Boo/Diva (44, 45), CED-9 (46), and KSbcl-2 from human herpes virus 8 (47), although the intracellular localization of these proteins and/or the precise role of this motif in targeting are unknown at present.

We have shown that M11L prevents mitochondria from undergoing a permeability transition after initiation of an apoptotic signal by staurosporine. We demonstrated this both in Rat2 cells stably expressing M11L and in HeLa cells transiently transfected with a GFP-M11L construct. Although there are pitfalls associated with the use of fluorescent dyes for monitoring mitochondrial function (48, 49), we obtained the same result using two different fluorescent probes in the two systems, supporting the conclusion that M11L protects mitochondria from apoptotic changes. In addition, the correct localization of M11L to mitochondria appears to be essential for this function. It is therefore likely that M11L acts as a viral survival effector by preventing amplification of apoptotic cascades that proceed via the mitochondrial pathway. Two other viral antiapoptotic proteins, herpes virus saimiri Bcl-2 and human CMV vMIA, both expressed by herpes viruses, are similarly important for preservation of mitochondrial function after exposure to apoptosis inducing agents (14, 15). In the case of myxoma virus and CMV, this function is also required to sustain viral replication (15, 23).

To investigate the apparently contradictory observation that the loss of M11L function results in the induction of both apoptosis and a massive inflammatory response in infected rabbits, we tested the ability of the M11L knockout virus, as well as various other deletion mutant viruses, to induce apoptosis during infection of primary rabbit monocytes. Whereas a number of these myxoma virus deletion mutants induce apoptosis in the RL-5 lymphocyte cell line, only the M11L knockout virus elicited an apoptotic response in primary rabbit monocytes. This indicates that expression of M11L might be particularly important in allowing myxoma virus to productively infect cells of the monocyte/macrophage lineage, and is interesting in view of the long-recognized importance of macrophages of the reticuloendothelial system in the development of myxomatosis (50). Elevated levels of apoptosis in tissues infected by the knockout virus would not be anticipated in association with signs of an increased inflammatory response except in the unusual situation of apoptosis in cells of the monocyte/macrophage lineage. This proinflammatory effect could be attributed to a variety of causes. Monocyte apoptosis has been shown to result in the processing and release of the inflammatory cytokine IL1b (38). In addition, monocyte-derived macrophages are responsible for limiting the inflammatory effects of activated neutrophils and other granulocytes. Hence, depletion of the monocyte population could impair the normal regulatory processes of the immune system, which are designed to contain the potentially dangerous effects of uncontrolled inflammation (37).

In conclusion, we show that M11L is an antiapoptotic protein that localizes to the exterior of mitochondria by means of a 25-amino acid COOH terminal targeting motif and protects this organelle from changes associated with apoptosis induction. M11L may be particularly important for circumventing an apoptotic response in monocytes/macrophages that infiltrate into lesions during myxoma virus infection of rabbits. It will now be of interest to investigate the role of M11L in protecting cells from additional apoptotic inducers and to ascertain in more detail the role of this protein in modulating mitochondrial function.

### REFERENCES CITED IN EXAMPLE 1

1. O'Brien, V. 1998. Viruses and apoptosis. J. Gen. Virol. 79: 1833-1845.

2. Tschopp, J., M. Thome, K. Hofmann, and E. Meinl. 1998. The fight of viruses against apoptosis. Curr. Opin. Genet. Dev. 8:82-87.

3. Everett, H., and G. McFadden. 1999. Apoptosis: an innate immune response to virus infection. Trends Microbiol. 7:160-165.

4. Barry, M., and G. McFadden. 1998. Apoptosis regulators from DNA viruses. Curr. Opin. Immunol. 10:422-430.

5. Hardwick, J. M. 1998. Viral interference with apoptosis. Semin. Cell Dev. Biol. 9:339-349.

6. Roulston, A., R. C. Marcellus, and P. E. Branton. 1999. Viruses and apoptosis. Annu. Rev. Microbiol. 53:577-628.

7. Meinl, E., H. Fickenscher, M. Thome, J. Tschopp, and B. Fleckenstein. 1998. Antiapoptotic strategies of lymphotropic viruses. Immunol. Today. 19:474-479.

8. Shisler, J. L., and L. R. Gooding. 1998. Adenoviral inhibitors of the apoptotic cascade. Trends Microbiol. 6:337-339.

9. Deveraux, Q. L., and J. C. Reed. 1999. IAP family proteins—suppressors of apoptosis. Genes Dev. 13:239-252.

10. Green, D. R., and J. C. Reed. 1998. Mitochondria and apoptosis. Science. 281:1309-1312.

11. Gross, A., J. M. McDonnell, and S. J. Korsmeyer. 1999. BCL-2 family members and the mitochondria in apoptosis. Genes Dev. 13:1899-1911.

12. Kroemer, G., B. Dallaporta, and M. RescheRigon. 1998. The mitochondrial death/life regulators in apoptosis and necrosis. Annu. Rev. Physiol. 60:619-642.

13. Vander Heiden, M. G., and C. B. Thompson. 1999. Bcl-2 proteins: regulators of apoptosis or of mitochondrial homeostasis? Nat. Cell Biol. 1:209-214.

14. Derfuss, T., H. Fickenscher, M. S. Kraft, G. Henning, D. Lengenfelder, B. Fleckenstein, and E. Meinl. 1998. Antiapoptotic activity of the herpes virus saimirien-coded Bcl-2 homolog: stabilization of mitochondria and inhibition of Caspase-3like activity. J. Virol. 72:5897-5904.

15. Goldmacher, V. S., L. M. Bartle, A. Skaletskaya, C. A. Dionne, N. L. Kedersha, C. A. Vater, J. W. Han, R. J. Lutz, S. Watanabe, E. D. McFarland, et al. 1999. A cytomegalovirus-encoded mitochondria-localized inhibitor of apoptosis structurally unrelated to Bcl-2. Proc. Natl. Acad. Sci. USA. 96: 12536-12541.

16. McFadden, G., and M. Barry. 1998. How poxviruses oppose apoptosis. Semin. Virol. 8:429-442.

17. Nash, P., J. Barrett, J. X. Cao, S. HotaMitchell, A. Lalani, H. Everett, X. M. Xu, J. Robichaud, S. Hnatiuk, C. Ainslie, et al. 1999. Immunomodulation by viruses: the myxoma virus story. Immunol. Rev. 168:103-120.

18. DiGiacomo, R. F., and C. J. Maré. 1994. Viral diseases. In The Biology of the Laboratory Rabbit. 2nd ed. P. J. Manning, D. H. Ringler, and C. E. Newcomer, editors. Academic Press, New York/London. 171-204.

19. Barry, M., S. Hnatiuk, K. Mossman, S. F. Lee, L. Boshkov, and G. McFadden. 1997. The myxoma virus M-T4 gene encodes a novel RDEL-containing protein that is retained within the endoplasmic reticulum and is important for the productive infection of lymphocytes. Virology. 239:360-377.

20. Macen, J. L., K. A. Graham, S. F. Lee, M. Schreiber, L. K. Boshkov, and G. McFadden. 1996. Expression of the myxoma virus tumor necrosis factor receptor homologue (T2) and M11L genes is required to prevent virus-induced apoptosis in infected rabbit T lymphocytes. Virology. 218:232-237.

21. Mossman, K., S. F. Lee, M. Barry, L. Boshkov, and G. McFadden. 1996. Disruption of M-T5, a novel myxoma virus gene member of the poxvirus host range superfamily, results in dramatic attenuation of myxomatosis in infected European rabbits. J. Virol. 70:4394-4410.

22. Graham, K. A., A. Opgenorth, C. Upton, and G. McFadden. 1992. Myxoma virus M11L ORF encodes a protein for which cell surface localization is critical for manifestation of viral virulence. Virology. 191:112-124.

23. Opgenorth, A., K. Graham, N. Nation, D. Strayer, and G. McFadden. 1992. Deletion analysis of two tandemly arranged virulence genes in myxoma virus, M11L and myxoma growth factor. J. Virol. 66:4720-4731.

24. Upton, C., J. L. Macen, M. Schreiber, and G. McFadden. 1991. Myxoma virus expresses a secreted protein with homology to the tumor necrosis factor receptor gene family that contributes to viral virulence. Virology. 184:370-382.

25. Macen, J. L., C. Upton, N. Nation, and G. McFadden. 1993. SERP1, a serine proteinase inhibitor encoded by myxoma virus, is a secreted glycoprotein that interferes with inflammation. Virology. 195:348-363.

26. Morgenstern, J. P., and H. Land. 1990. Advanced mammalian gene transfer: high titer retroviral vectors with multiple drug selection markers and a complementary helper-free packaging cell line. Nucleic Acids Res. 18:3587-3596.

27. Pear, W. S., G. P. Nolan, M. L. Scott, and D. Baltimore. 1993. Production of high-titer helper-free retroviruses by transient transfection. Proc. Natl. Acad. Sci. USA. 90:8392-8396.

28. Evans, K., K. Schifferli, and P. HawleNelson. 1999. High-efficiency transfection of HeLa cells. Focus. 21:15.

29. Sgonc, R., G. Boeck, H. Dietrich, J. Gruber, H. Recheis, and G. Wick. 1994. Simultaneous determination of cell surface antigens and apoptosis. Trends Genet. 10:41-42.

30. Atkinson, E. A., M. Barry, A. J. Darmon, I. Shostak, P. C. Turner, R. W. Moyer, and R. C. Bleackley. 1998. Cytotoxic T lymphocyte-assisted suicide. J. Biol. Chem. 273:21261-21266.

31. Nguyen, M., D. G. Millar, V. W. Youg, S. J. Korsmeyer, and G. C. Shore. 1993. Targeting of Bcl-2 to the mitochondrial outer membrane by a COOH terminal signal anchor sequence. J. Biol. Chem. 268:25265-25268.

32. Metivier, D., B. Dallaporta, N. Zamzami, N. Larochette, S. A. Susin, I. Marzo, and G. Kroemer. 1998. Cytofluorometric detection of mitochondrial alterations in early CD95/Fas/Apo1triggered apoptosis of Jurkat T lymphoma cells. Comparison of seven mitochondrion-specific fluorochromes. Immunol. Lett. 61:157-163.

33. Ehrenberg, B., V. Montana, M. D. Wei, J. P. Wuskell, and L. J. Loew. 1988. Membrane potential can be determined in individual cells from the Nernstian distribution of cationic dyes. Biophys. J. 53:785-794.

34. Farkas, D. L., M. Wei, P. Febbroriello, J. H. Carson, and L. M. Loew. 1989. Simultaneous imaging of cell and mitochondrial membrane potential. Biophys. J. 56:1053-1069.

35. Lazebnik, Y. A., S. H. Kaufmann, S. Desnoyers, G. G. Poirier, and W. C. Eamshaw. 1994. Cleavage of poly(ADPribose) polymerase by a proteinase with properties like ICE. Nature. 371:346-347.

36. Isenmann, S., Y. KhewGoodall, J. Gamble, M. Vadas, and B. W. Wattenberg. 1998. A splice isoform of vesicle-associated membrane protein1 (VAMP1) contains a mitochondrial targeting signal. Mol. Biol. Cell. 9:1649-1660.

37. Savill, J. 1997. Apoptosis in the resolution of inflammation. J. Leukoc. Biol. 61:375-380.

38. Hogquist, K., M. A. Nett, E. R. Unanue, and D. D. Chaplin. 1991. Interleukin 1 is processed and released during apoptosis. Proc. Natl. Acad. Sci. USA. 88:8485-8489.

39. Janiak, F., B. Leber, and D. W. Andrews. 1994. Assembly of Bcl-2 into microsomal and outer mitochondrial membranes. J. Biol. Chem. 269:9842-9849.

40. Wolter, K. G., Y. T. Hsu, C. L. Smith, A. Nechushtan, X. G. Xi, and R. J. Youle. 1997. Movement of Bax from the cytosol to mitochondria during apoptosis. J. Cell Biol. 139: 1281-1292.

41. Yasuda, M., P. Theodorakis, T. Subramanian, and G. Chinnadurai. 1998. Adenovirus E1B19K/Bcl-2 interacting protein BNIP3 contains a BH3 domain and a mitochondrial targeting sequence. J. Biol. Chem. 273:12415-12421.

42. Chen, B. G., R. Ray, D. Dubik, L. Shi, J. Cizeau, R. C. Bleackley, S. Saxena, R. D. Geitz, and A. H. Greenberg. 1997. The E1B 19k/Bcl-2 binding protein Nip 3 is a dimeric mitochondrial protein that activates apoptosis. J. Exp. Med. 186:1975-1983.

43. Chen, G., J. Cizeau, C. Vande Velde, J. H. Park, G. Bozek, J. Bolton, L. Shi, D. Dubik, and A. Greenberg. 1999. Nix and Nip3 form a subfamily of proapoptotic mitochondrial proteins. J. Biol. Chem. 274:7-10.

44. Song, Q., Y. Kuang, V. Dixit, and C. Vincenz. 1999. Boo, a novel negative regulator of cell death, interacts with Apaf1. EMBO (Eur. Mol. Biol. Organ.) J. 18:167-178.

45. Inohara, N., T. S. Gourley, R. Carrio, M. Muniz, J. Merino, I. Garcia, T. Koseki, T. Hu, S. Chen, and G. Nunez. 1998. Diva, a Bcl-2 homolog that binds directly to Apaf1 and induces BH3independent cell death. J. Biol. Chem. 273: 32479-32486.

46. Wu, D., H. D. Wallen, and G. Nunez. 1997. Interaction and regulation of subcellular localization of CED4 by CED-9. Science. 275:1126-1129.

47. Cheng, E. H. Y., J. Nicholas, D. S. Bellows, G. S. Hayward, H. G. Guo, M. S. Reitz, and J. M. Hardwick. 1997. A Bcl-2 homolog encoded by Kaposi sarcoma-associated virus, human herpes virus 8, inhibits apoptosis but does not heterodimerize with BAX or BAK. Proc. Natl. Acad. Sci. USA. 94: 690-694.

48. Bernardi, P., L. Scorrano, R. Colonna, V. Petronilli, and F. Di Lisa. 1999. Mitochondria and cell death. Eur. J. Biochem. 264:687-701.

49. Poot, M., and R. C. Pierce. 1999. Detection of apoptosis and changes in mitochondrial membrane potential with chloromethyl xrosamine. Cytometry. 36:359-360.

50. Ahlstrom, C. G. 1940. On the anatomical character of the infectious myxoma of rabbits. Acta Pathol. 18:377-393.

### EXAMPLE 2

Apoptosis Inhibition by Poxviruses

All viruses must learn how to modulate apoptosis in order to survive in the widely diverse cell types they typically encounter within an infected host. To date, all of the known modulators of apoptosis encoded by poxviruses are inhibitors. Mechanistically, they are targeted to a broad variety of cellular locations, ranging from the cytoplasm, the ER and the mitochondria. M11L, a 166 a.a. protein produced by the Myxoma poxvirus, is required to prevent apoptosis of virus-infected leukocytes and also plays an anti-apoptotic role when expressed autonomously from other viral proteins. We have shown that M11L targets to the mitochondria and functions upstream of Caspase-3 activation and cytochrome c release but downstream of Bid cleavage. This provides evidence that M11L impacts apoptotic cascades by modulating the mitochondrial control point of cell death. This view is further supported by the finding that M11L is localized to the cytoplasmic aspect of mitochondria by a short 25 a.a. C-terminal targeting motif. A point mutation in this motif redirects the M11L variant protein to other intracellular membranes, notably the endoplasmic reticulum. M11L can protect mitochondria from undergoing loss of inner membrane potential, an event frequently associated with apoptosis and experiments to determine how this is achieved are currently in progress. M11L was immunoprecipitated from HEK293 cells and interacting proteins were identified by mass spectrometry analysis. BAK, an apoptotic member of the Bcl-2 family, was identified as an M11L-binding partner. BAK functions to accelerate apoptosis by binding to and antagonizing Bcl-2. These results are consistent with a role for M11L at the mitochondria downstream of Bid cleavage.

Anti-apoptotic viral genes like M11L provide a novel resource with which to engineer vectors that resist immune clearance by CTLs and NK cells, and which can be potentially exploited for both gene therapy and vaccine vector development.

Cloning

The M11L coding sequence was PCR amplified from the plasmid pMYS2a (1) and cloned into the T-tailed cloning vector, pT7Blue (Novagen) using the a T-tailed cloning kit (Novagen) to generate the plasmid pT7M11L. M11L was reamplified from pT7M11L using the primers:

| | |
|---|---|
| 5′ M11L FLAG: | |
| (5′-ATGGACTACAAGGACGACGATGACAAGTCTCGTTTAAAGACGGCCG-3′, and | SEQ ID No. 6) |
| 3′ M11L SalI: | |
| (5′-CGTAgtcgacTAGGTCCCTCGGTACC-3′,. | SEQ ID No. 7) |

The M11L coding sequence with the 5′FLAG sequence appended to the 5′ end was cloned into the vector pT7Blue-3 (Novagen) using the Perfectly Blunt kit (Novagen) and the construct verified by sequencing. The coding sequence was cloned as a HindIII-BamHI fragment into pcDNA3 to generate pcDNA3 M11L 5′FLAG. The construct was again verified by sequencing.

Cell Culture and Ectopic Expression

HEK293T cells were grown in DMEM media (Gibco-BRL) with 10% FBS (Sigma-Aldrich), 1% 1-glutamine (Gibco-BRL), and 1% non-essential amino acids (Gibco-BRL). HEK293T cells (20 10-cm-diameter plates) were transfected with Flag-M11L DNA by CaPO₄ precipitation (Stone et al. (1991) Methods Enzymol 200:673-692.).

Immunoprecipitation

Approximately 44 hours after transfection, 1×10⁸ cells (20 10-cm-diameter plates) each of HEK293T cells transfected with Flag-M11L and control HEK293T cells were washed in Tris-Saline (25 mM Tris-HCl pH 7.5, 140 mM NaCl, 8 mM KCl, 700 μM Na₂HP0₄, 5.5 mM glucose) and lysed in kinase lysis buffer (20 mM Tris-HCl pH7.5, 150 mM NaCl, 1% NP-40, 0.5% sodium deoxycholate, 0.2 mM AEBSF (4-(2-aminoethyl)-benzenesulfonyl fluoride), 1.5 μg/ml aprotinin, 1 μg/ml leupeptin, and 2 μM pepstatin). Clarified lysates were incubated with sepharose 4B (10 μL packed sepharose/ml of lysate, Sigma-Aldrich) for 20 minutes at 4° C. with gentle rotation. The supernatant was then incubated with M2-agarose (1 μl packed M2-agarose/ml lysate, Sigma-Aldrich) for 60 minutes at 4° C. with gentle rotation. The M2-agarose was washed two times with 1 ml of kinase lysis buffer, and washed one time with 1 ml of 50 mM ammonium bicarbonate. The M2-agarose was incubated with 400 μg/ml Flag peptide (Sigma-Aldrich) in 50 mM ammonium bicarbonate to elute the Flag-tagged protein. The eluted proteins were lyophilized by vacuum centrifugation and resuspended in 10 μl H₂O.

Preparation of Sample and SDS-PAGE

The resuspended sample was boiled in a final concentration of 1× SDS sample buffer pH 8.8 (Maniatis, T., Fritsch, E. F. and Sambrook, J. (Eds.) Molecular Cloning—a laboratory manual, Cold Spring Harbor: Cold Spring Harbor Laboratory, 1982). Samples were loaded onto a 4-15% acrylamide Tris-HCl gel from BioRad. The gels were stained with Pierce GELCODE blue stain reagent according to the manufacturers instructions. Bands present in both the Flag-M11L IP and the control HEK293T IP were excised and prepared for mass spectrometer analysis.

Mass Spectrometry

The proteins were reduced, the free cysteine residues were alkylated with iodoacetamide, and the proteins were subjected to digestion by trypsin (Promega) using the method of Shevchenko et al. (1996, Anal. Chem 68:850-858). The extracted peptides were purified by C₁₈ reverse-phase chromatography and resuspended in 5% methanol-5% formic acid prior to analysis. Mass spectrometry was carried out on a quadrapole-time-of-flight hybrid mass spectrometer (Sciex QSTAR; see Shevchenko et al. (1997) Rapid Commun. Mass Spectrom 11:1015-1024) equipped with a nanospray ion source (Protana). Each sample was introduced into a nanospray needle installed in front of the mass spectrometer orifice and continuously electro-sprayed at a low flow rate as previously described (Wilm et al. (1996) Anal. Chem. 68:1-8). MS spectra were acquired to determine the m/z ratios of the peptides present in the proteolytic digest. Individual peptides were selected for fragmentation by collision-induced dissociation and the resulting fragments separated, generating an MS-MS spectrum. For every MS-MS spectrum, a small stretch of the amino acid sequence was manually determined, generating a sequence tag, which was fed into a search engine (PeptideScan). The search engine was used to identify the provenance of the peptide using protein-DNA database searches. Every peptide identified was confirmed manually.

We claim:

1. A method for identifying agents which may be potentially pro-apoptotic or anti-apoptotic, comprising:
A) providing a reaction mixture including complexes of BAK and/or M11L proteins;
B) contacting the reaction mixture with one or more test agents;
C) determining if the test agent possesses at least one of the following abilities:
1) binding to the complex;
2) increasing or decreasing the steady state level of the complex;
3) affecting an enzymatic activity of the complex;
4) affecting a subcellular localization of the complex.

2. The method of claim 1, wherein the agent is polypeptides, nucleic acids, carbohydrates, small organic molecules, or natural product extract libraries.

3. The method of claim 2, wherein the agent is from natural product extract libraries isolated form animals, plants, fungus, or microbes.

4. The method of claim 1, wherein the method is repeated for a variegated library of at least 10 different members.

5. The method of claim 4, wherein the method is repeated for a variegated library of at least 100 different members.

6. The method of claim 5, wherein the method is repeated for a variegated library of at least 1,000 different members.

7. The method of claim 6, wherein the method is repeated for a variegated library of at least 10,000 different members.

8. The method of claim 1, further comprising:
D) determining if the test agent, which possesses at least one of the abilities of C), is pro-apoptotic or anti-apoptotic.

9. The method of 8, wherein step D) is carried out in vivo or in whole cells.

10. The method of claim 1, wherein the reaction mixture is a cell-free system.

11. The method of claim 10, wherein the cell-free system comprises reconstituted protein mixture of semi-purified proteins.

12. The method of claim 10, wherein the cell-free system comprises reconstituted protein mixture of highly-purified proteins substantially lacking impurity.

13. The method of claim 10, wherein at least one member of said complexes of BAK and/or M11L proteins, or the test agent, is immobilized on a solid support.

14. The method of claim 13, wherein the immobilization is effected by chemical cross-linking, by indirect conjugating via an intermediate molecule, or by direct coating of said solid support.

15. The method of claim 14, wherein the intermediate molecule is an antibody or biotin.

16. The method of claim 13, wherein the solid support is microtiter plates, microarrays, test tubes, microcentrifuge tubes, or solid matrices.

17. The method of claim 13, wherein said at least one member of said complexes of BAK and/or M11L proteins, or said test agent is a fusion protein adapted to bind said solid support.

18. The method of claim 10, wherein at least one member of said complexes of BAK and/or M11L proteins, or the test agent, is labeled.

19. The method of claim 18, wherein the label is a radioisotope, a fluorescent label/tag, an epitope tag, or an enzyme.

20. The method of claim 10, wherein the cell-free system is generated from lysates, each containing one or more of the relevant polypeptides, which lysates are mixed appropriately or spiked, wherein no single lysate contains all the component necessary for generating said cell-free system.

21. The method of claim 20, wherein one or more of said relevant polypeptides is recombinantly generated.

22. The method of claim 20, wherein said lysates derive from one or more cell types selected from bacteria cells, yeast cells, worm cells, insect cells, amphibian cells, plant cells, or mammalian cells.

23. The method of claim 1 or 8, wherein the reaction mixture is a cell.

24. The method of claim 23, wherein the method is carried out using a yeast two-hybrid assay or reverse yeast two-hybrid assay.

25. The method of claim 24, wherein the method employs an Interaction Trap System (ITS) or reverse ITS.

26. A method of conducting a drug discovery business comprising:
A) providing one or more assay systems for identifying agents by their ability to inhibit or potentiate BAK-dependent and/or M11L-dependent apoptosis;
B) conducting therapeutic profiling of agents identified in step A), or further analogs thereof, for efficacy and toxicity in animals; and
C) formulating a pharmaceutical preparation including one or more agents identified in step (B) as having an acceptable therapeutic profile.

27. The method of claim 26, further comprising a step of establishing a distribution system for distributing the pharmaceutical preparation for sale.

28. The method of claim 26, further comprising a step of establishing a sales group for marketing the pharmaceutical preparation.

29. A method of conducting a target discovery business comprising:
A) providing one or more assay systems for identifying agents by their ability to inhibit or potentiate BAK-dependent and/or M11L-dependent apoptosis;
B) (optionally) conducting therapeutic profiling of agents identified in step A) for efficacy and toxicity in animals; and
C) licensing, to a third party, the rights for further drug development and/or sales for gents identified in step A), or analogs thereof.

30. A method to identify M11L-interacting polypeptides, comprising:
A) contacting M11L with a reaction mixture;
B) retrieving M11L, together with any M11L-interacting polypeptides, from the reaction mixture;
C) identifying M11L-interacting polypeptides of B) using mass spectrometry.

31. The method of claim 30, wherein M11L is a recombinant protein.

32. The method of claim 30, wherein M11L is provided as a fusion protein.

33. The method of claim 30, wherein the reaction mixture is a cell-free system.

34. The method of claim 30, wherein the reaction mixture is a cell.

35. The method of claim 30, wherein step B) is effected by immunoprecipitation.

36. The method of claim 30, further comprising separating M11L from M11L-interacting polypeptides before step C).

37. The method of claim 36, further comprising digesting separated M11L-interacting polypeptides before step C).
